SEQUENCE LISTING (14 sequences)

SEQ ID NO: 1 - DNA, 21 bp, synthetic construct
  misc_feature 1..21: note = CD28 target sequence 1
  source 1..21: mol_type = other DNA
caaccttagctgcaagtattc

SEQ ID NO: 2 - DNA, 21 bp, synthetic construct
  misc_feature 1..21: note = CD28 target sequence 2
  source 1..21: mol_type = other DNA
tggagtcctggcttgctatag

SEQ ID NO: 3 - DNA, 21 bp, synthetic construct
  misc_feature 1..21: note = CD28 target sequence 3
  source 1..21: mol_type = other DNA
cctcctccttacctagacaat

SEQ ID NO: 4 - DNA, 21 bp, synthetic construct
  misc_feature 1..21: note = CD28 target sequence 4
  source 1..21: mol_type = other DNA
gctgtggaagtctgtgttgta

SEQ ID NO: 5 - DNA, 21 bp, synthetic construct
  misc_feature 1..21: note = CD28 target sequence 5
  source 1..21: mol_type = other DNA
cgcaagcattaccagccctat

SEQ ID NO: 6 - DNA, 21 bp, synthetic construct
  misc_feature 1..21: note = CD28 target sequence 6
  source 1..21: mol_type = other DNA
cttcaattcaagtaacaggaa

SEQ ID NO: 7 - DNA, 2264 bp, Homo sapiens
  source 1..2264: mol_type = genomic DNA
agtttcccacaagccaggctgatccttttctgtcagtccacttcaccaagcctgcccttggacaaggacccgatgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctgacctcaagatcaaggaaaggaggatggacgaacaggggccaaactggtgggaggcagaggtggtgggggcagggatgataggccctggatgtgcccacagggaccaagaagtgaggtttccactgtcttgcctgccagggcccctgttcccccgctggcagccaccccctcccccatcatatcctttgccccaaggctgctcagaggggccccggtcctggccccagcccccacctccgccccagacacaccccccagtcgagccctgcagccaaacagagccttcacaaccagccacacagagcctgcctcagctgctcgcacagattacttcagggctggaaaagtcacacagacacacaaaatgtcacaatcctgtccctcactcaacacaaaccccaaaacacagagagcctgcctcagtacactcaaacaacctcaaagctgcatcatcacacaatcacacacaagcacagccctgacaacccacacaccccaaggcacgcacccacagccagcctcagggcccacaggggcactgtcaacacaggggtgtgcccagaggcctacacagaagcagcgtcagtaccctcaggatctgaggtcccaacacgtgctcgctcacacacacggcctgttagaattcacctgtgtatctcacgcatatgcacacgcacagccccccagtgggtctcttgagtcccgtgcagacacacacagccacacacactgccttgccaaaaataccccgtgtctcccctgccactcacctcactcccattccctgagccctgatccatgcctcagcttagactgcagaggaactactcatttatttgggatccaaggcccccaacccacagtaccgtccccaataaactgcagccgagctcccca

SEQ ID NO: 8 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 9 - DNA, 876 bp, Homo sapiens
  source 1..876: mol_type = genomic DNA
atggccacaaccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagtcatcgatatcctcgaggtcaccgcggtctagagtcgacctgcagccaagcttatcgataa

SEQ ID NO: 10 - protein, 277 aa, Homo sapiens [sequence omitted]

SEQ ID NO: 11 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 12 - DNA, 4430 bp, Homo sapiens
  source 1..4430: mol_type = genomic DNA
acacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaagaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactactatatgtctgctttaaatttgtactttaatattgtcttttggtattaagaaagatatgctttcagaatagatatgcttcgctttggcaaggaatttggatagaacttgctatttaaaagaggtgtggggtaaatccttgtataaatctccagtttagccttttttgaaaaagctagactttcaaatactaatttcacttcaagcagggtacgtttctggtttgtttgcttgacttcagtcacaatttcttatcagaccaatggctgacctctttgagatgtcaggctaggcttacctatgtgttctgtgtcatgtgaatgctgagaagtttgacagagatccaacttcagccttgaccccatcagtccctcgggttaactaactgagccaccggtcctcatggctattttaatgagggtattgatggttaaatgcatgtctgatcccttatcccagccatttgcactgccagctgggaactataccagacctggatactgatcccaaagtgttaaattcaactacatgctggagattagagatggtgccaataaaggacccagaaccaggatcttgattgctatagacttattaataatccaggtcaaagagagtgacacacactctctcaagacctggggtgagggagtctgtgttatctgcaaggccatttgaggctcagaaagtctctctttcctatagatatatgcatactttctgacatataggaatgtatcaggaatactcaaccatcacaggcatgttcctacctcagggcctttacatgtcctgtttactctgtctagaatgtccttctgtagatgacctggcttgcctcgtcacccttcaggtccttgctcaagtgtcatcttctcccctagttaaactaccccacaccctgtctgctttccttgcttatttttctccatagcattttaccatctcttacattagacatttttcttatttatttgtagtttataagcttcatgaggcaagtaactttgctttgtttcttgctgtatctccagtgcccagagcagtgcctggtatataataaatatttattgactgagtgaa

SEQ ID NO: 13 - DNA, 4543 bp, Homo sapiens
  source 1..4543: mol_type = genomic DNA
taaagtcatcaaaacaacgttatatcctgtgtgaaatgctgcagtcaggatgccttgtggtttgagtgccttgatcatgtgccctaaggggatggtggcggtggtggtggccgtggatgacggagactctcaggccttggcaggtgcgtctttcagttcccctcacacttcgggttcctcggggaggaggggctggaaccctagcccatcgtcaggacaaagatgctcaggctgctcttggctctcaacttattcccttcaattcaagtaacagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaagaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactactatatgtctgctttaaatttgtactttaatattgtcttttggtattaagaaagatatgctttcagaatagatatgcttcgctttggcaaggaatttggatagaacttgctatttaaaagaggtgtggggtaaatccttgtataaatctccagtttagccttttttgaaaaagctagactttcaaatactaatttcacttcaagcagggtacgtttctggtttgtttgcttgacttcagtcacaatttcttatcagaccaatggctgacctctttgagatgtcaggctaggcttacctatgtgttctgtgtcatgtgaatgctgagaagtttgacagagatccaacttcagccttgaccccatcagtccctcgggttaactaactgagccaccggtcctcatggctattttaatgagggtattgatggttaaatgcatgtctgatcccttatcccagccatttgcactgccagctgggaactataccagacctggatactgatcccaaagtgttaaattcaactacatgctggagattagagatggtgccaataaaggacccagaaccaggatcttgattgctatagacttattaataatccaggtcaaagagagtgacacacactctctcaagacctggggtgagggagtctgtgttatctgcaaggccatttgaggctcagaaagtctctctttcctatagatatatgcatactttctgacatataggaatgtatcaggaatactcaaccatcacaggcatgttcctacctcagggcctttacatgtcctgtttactctgtctagaatgtccttctgtagatgacctggcttgcctcgtcacccttcaggtccttgctcaagtgtcatcttctcccctagttaaactaccccacaccctgtctgctttccttgcttatttttctccatagcattttaccatctcttacattagacatttttcttatttatttgtagtttataagcttcatgaggcaagtaactttgctttgtttcttgctgtatctccagtgcccagagcagtgcctggtatataataaatatttattgactgagtgaaaaaaaaaaaaaaaaa

SEQ ID NO: 14 - DNA, 4721 bp, Homo sapiens
  source 1..4721: mol_type = genomic DNA
acacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaagaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactactatatgtctgctttaaatttgtactttaatattgtcttttggtattaagaaagatatgctttcagaatagatatgcttcgctttggcaaggaatttggatagaacttgctatttaaaagaggtgtggggtaaatccttgtataaatctccagtttagccttttttgaaaaagctagactttcaaatactaatttcacttcaagcagggtacgtttctggtttgtttgcttgacttcagtcacaatttcttatcagaccaatggctgacctctttgagatgtcaggctaggcttacctatgtgttctgtgtcatgtgaatgctgagaagtttgacagagatccaacttcagccttgaccccatcagtccctcgggttaactaactgagccaccggtcctcatggctattttaatgagggtattgatggttaaatgcatgtctgatcccttatcccagccatttgcactgccagctgggaactataccagacctggatactgatcccaaagtgttaaattcaactacatgctggagattagagatggtgccaataaaggacccagaaccaggatcttgattgctatagacttattaataatccaggtcaaagagagtgacacacactctctcaagacctggggtgagggagtctgtgttatctgcaaggccatttgaggctcagaaagtctctctttcctatagatatatgcatactttctgacatataggaatgtatcaggaatactcaaccatcacaggcatgttcctacctcagggcctttacatgtcctgtttactctgtctagaatgtccttctgtagatgacctggcttgcctcgtcacccttcaggtccttgctcaagtgtcatcttctcccctagttaaactaccccacaccctgtctgctttccttgcttatttttctccatagcattttaccatctcttacattagacatttttcttatttatttgtagtttataagcttcatgaggcaagtaactttgctttgtttcttgctgtatctccagtgcccagagcagtgcctggtatataataaatatttattgactgagtgaa

(19) United States
(12) Patent Application Publication
(10) Pub. No.: US 2023/0330141 A1
(43) Pub. Date: Oct. 19, 2023

(54) METHODS OF PRODUCING T REGULATORY CELLS, METHODS OF TRANSDUCING T CELLS, AND USES OF THE SAME

(71) Applicant: Kyverna Therapeutics, Inc., Emeryville, CA (US)

(72) Inventors: Mamle Quarmyne, Emeryville, CA (US); Faye Wu, Emeryville, CA (US); Jordan Tsai, Emeryville, CA (US); John Lee, Emeryville, CA (US); Jeffrey Greve, Emeryville, CA (US); Joseph Cheng, Emeryville, CA (US); Meghana Vijayraghavan, Emeryville, CA (US)

(73) Assignee: Kyverna Therapeutics, Inc., Emeryville, CA (US)

(21) Appl. No.: 17/985,587

(22) Filed: Nov. 11, 2022

Related U.S. Application Data
(63) Continuation of application No. 17/391,767, filed on Aug. 2, 2021.
(60) Provisional application No. 63/158,722, filed on Mar. 9, 2021.
(60) Provisional application No. 63/060,388, filed on Aug. 3, 2020.

(51) Int. Cl. A61K 35/17; C07K 14/725; C07K 16/28; C12N 15/113

(52) U.S. Cl. CPC A61K 35/17; C07K 14/7051; C07K 16/2818; C12N 15/113; A61K 38/00

(57) ABSTRACT

This document relates to methods and materials for treating a mammal having an autoimmune disease. For example, materials and methods for producing a T cell comprising a FOXP3 polypeptide. Methods and materials for treating a mammal having an autoimmune disease comprising administering to a mammal having an autoimmune disease an effective amount of a T cell are also provided herein.

### CROSS-REFERENCE TO RELATED APPLICATIONS

This application is a continuation of U.S. patent application Ser. No. 17/391,767, filed on Aug. 2, 2021, which claims priority to U.S. Provisional Patent Application No. 63/060,388, filed on Aug. 3, 2020, and U.S. Provisional Patent Application No. 63/158,722 filed on Mar. 9, 2021, the entire contents of each of these applications is incorporated herein by reference.

### SEQUENCE LISTING

This application contains a Sequence Listing that has been submitted electronically as an XML file named 47902-0028002.xml. The XML file, created on Nov. 11, 2022, is 33,207 bytes in size. The material in the XML file is hereby incorporated by reference in its entirety.

### BACKGROUND

Autoimmunity is a common disease in the United States, with more than 20 million people suffering from one of 81 known autoimmune diseases. Regulatory T cells (Tregs) are a subpopulation of T cells that modulate the immune system and maintain tolerance to self-antigens. Tregs play a role in preventing or treating autoimmune disease (Sakaguchi et al., Int'l Immun., 21(10):1105-1111 (2009)). FOXP3, a transcription factor expressed in Tregs, has been implicated in specifying the phenotype of Tregs and maintaining Treg immunosuppressive functions (Hori et al., Science, 299:1057-61 (2003); Fontenot, et al., Nat Immunol., 4:330-6 (2003); Khattri, et al., Nat Immunol., 4:337-42 (2003)). Treg activation can include three signals: (1) TCR activation, (2) costimulatory receptor activation, and (3) cytokine signaling. The cytokine IL-2 provides the cytokine signal for Tregs.

Some Tregs are produced in the thymus from T cell precursors. These cells are referred to as tTregs. tTregs are thought to be directed to self-antigens that are encountered in the thymus during their development. Tregs can be generated in the periphery from mature conventional CD4 T cells (Tconv) as a result of antigen stimulation in the presence of IL-2 and TGF-β. These Tregs are referred to as peripherally-derived Tregs, or pTregs. The term iTreg, or induced Tregs, is used to describe cells that are artificially produced in vitro by treatment of Tconv cells with a TCR activator (such as antigen, or immobilized anti-CD3 antibody), IL-2, and/or TGF-β. iTregs share many phenotypic characteristics of tTregs and pTregs, but unlike tTregs and pTregs, are unstable and can revert to effector T cells (Chen et al., J. Exp. Med., 198: 1875-1886 (2003)).

FOXP3⁺ Tregs suppress the function of effector T cells and other immune cells causing autoimmune disease by a variety of immunosuppressive mechanisms, including by promoting IL-2 consumption through the high affinity TL-2 receptor, by blocking CD28 costimulatory signals through CTLA4, by production of Adenosine through the action of CD39 and CD73, by secretion of the immunosuppressive cytokine IL-10, as well as other mechanisms. Like all T cells, Tregs possess T Cell Receptors (TCRs) and are specific for certain antigens. Adoptive transfer of Tregs have been shown to be efficacious in a number of animal models of autoimmune disease, and adoptive transfer of Tregs is being evaluated in a number clinical trials in human for autoimmune diseases, for transplantation, and for graft-vs-host disease (Ferreira, et al., Nat Rev Drug Discov., 18:749-769 (2019)). However, it has proven difficult to manufacture natural Tregs in the numbers required to provide an efficacious dose for human patient. Factors that impact the manufacturability include the relatively low frequency of Tregs in peripheral blood relative to other immune cells, specialized growth conditions required for Tregs ex vivo, contamination of Tregs by Tconv cells, and ability to purify expanded Tregs. Therefore, developing methods of producing Tregs by genetic reprogramming of a more abundant immune cell type is of high interest and of great potential.

This document relates to methods and materials for treating a mammal having an autoimmune disease. For example, this document provides materials and methods for producing a T cell comprising a forkhead box P3 (FOXP3) polypeptide. This document also provides methods and materials for treating a mammal having an autoimmune disease, where the methods include administering to a mammal having an autoimmune disease an effective amount of the T regulatory cell.

### SUMMARY

This document provides methods and materials that can be used to produce T regulatory (Treg) cells that can be used, e.g., to treat mammals identified as having an autoimmune disease. The methods provided herein enable production of Treg cells harboring stable expression of an exogenous FOXP3 polypeptide. The Treg cells generated herein that stably express an exogenous FOXP3 polypeptide can be, e.g., used to treat mammals identified or diagnosed as having an autoimmune disease. In addition, the Treg cells generated herein can be further engineered to express other genes to enhance the beneficial properties and therapeutic activity of the cells.

Reprogramming non-Treg cells into Treg-like cells can be performed by transduction of a nucleic acid sequence encoding a FOXP3 polypeptide sequence to a T cell. In some embodiments, non-Treg cells include, without limitation, CD4⁺ conventional T cells (Tconv), they can be CD4⁺ CD45RA⁺ naïve Tconv cells, or CD3⁺ T cells. In some embodiments, transduction of T cells by lentivirus, an exemplary viral gene vector, can be performed after the T cell has been activated.

In one aspect, this disclosure features a method of producing T regulatory cells, including: contacting a T cell with an effective amount of (i) one or more CD3-stimulation agent(s) in the absence of a CD28 stimulating agent for a first period of time under conditions that allow for stimulation and activation of the T cell, and introducing into the T cell an effective amount of a nucleic acid sequence encoding a forkhead box protein 3 (FOXP3) polypeptide, where the presence of the nucleic acid sequence in the T cell induces the T cell to develop or further develop one or more characteristics of a T regulatory cell phenotype compared to when the nucleic acid sequence is not present in the T cell. In some embodiments, the method further includes contacting the T cell with an effective amount of one or more agent(s) that decreases CD28 expression and/or activity. In some embodiments, the method includes contacting a T cell with an effective amount of (i) one or more CD3-stimulation agent(s) in the absence of a CD28 stimulating agent for a first period of time under conditions that allow for stimulation and activation of the T cell before introducing into the T cell an effective amount of a nucleic acid sequence encoding a forkhead box protein 3 (FOXP3) polypeptide. In some embodiments, the method includes introducing into the T cell an effective amount of a nucleic acid sequence encoding a forkhead box protein 3 (FOXP3) polypeptide before contacting a T cell with an effective amount of (i) one or more CD3-stimulation agent(s) in the absence of a CD28 stimulating agent for a first period of time under conditions that allow for stimulation and activation of the T cell.

In another aspect, this disclosure features a method of producing T regulatory cells, including: contacting a T cell with an effective amount of (i) one or more CD3-stimulation agent(s), and (ii) one or more CD28-stimulation agent(s) for a first period of time under conditions that allow for stimulation and activation of the T cell; contacting the T cell with an effective amount of one or more agent(s) that decreases CD28 expression and/or activity; and introducing into the T cell an effective amount of a nucleic acid sequence encoding a forkhead box protein 3 (FOXP3) polypeptide, where the presence of the nucleic acid sequence in the T cell induces the T cell to develop or further develop one or more characteristics of a T regulatory cell phenotype compared to when the nucleic acid sequence is not present in the T cell.

In some embodiments, the method further includes contacting the T cell with an effective amount of interleukin-2 (IL-2) and/or TGF-β for a second period of time under conditions that allow for stabilization of a T regulatory phenotype as compared to when the T cell is not contacted with IL-2 and/or TGF-β for the second period of time. In some embodiments, the method does not include contacting the T cell with IL-2. In some embodiments, the method does not include contacting the T cell with TGF-β. In some embodiments, the method does not include contacting the T cell with IL-2 or TGF-β.

In some embodiments, the one or more CD3-stimulation agent(s) includes an effective amount of an anti-CD3 antibody. In some embodiments, the one or more CD3-stimulation agent(s) include a methyl transferase inhibitor.

In some embodiments, the one or more CD28-stimulation agents includes an anti-CD28 activating antibody.

In some embodiments, the one or more agent(s) that decreases CD28 expression and/or activity include an anti-CD28 blocking antibody. In some embodiments, the one or more agent(s) that decreases CD28 expression and/or activity include a small interfering RNA (siRNA) or a short hairpin RNA (shRNA). In some embodiments, the siRNA or the shRNA decreases expression of CD28 in a T cell. In some embodiments, the siRNA includes a sequence of one of SEQ ID NOs: 1-6. In some embodiments, the siRNA or shRNA decreases expression of one or more of p85, p110, PIP3, PKB/Akt, mTOR, IκB, GSK3β, NFκB, NFAT, LCK, FYN, and ITK in a T cell.

In some embodiments, the method further includes introducing into the T cell a nucleic acid construct including a sequence encoding the siRNA or the shRNA. In some embodiments, the nucleic acid construct further includes a promoter operably linked to the sequence encoding the shRNA.

In some embodiments, the one or more agent(s) that decreases CD28 expression and/or activity include a small molecule inhibitor of any one of: LCK, FYN, and ITK.

In some embodiments, the step of contacting of the T cell with an effective amount of one or more agent(s) that decreases CD28 expression and/or activity further includes removing the one or more agent(s) that decreases CD28 expression and/or activity after about 1 hour to about 60 hours of the first period of time.

In some embodiments, the method includes contacting a T cell with an effective amount of (i) one or more CD3-stimulation agent(s), and (ii) one or more CD28-stimulation agent(s) for a first period of time under conditions that allow for stimulation and activation of the T cell before performing the step of contacting the T cell with an effective amount of one or more agent(s) that decreases CD28 expression and/or activity and before performing the step of introducing into the T cell an effective amount of a nucleic acid sequence encoding a forkhead box protein 3 (FOXP3) polypeptide. In some embodiments, the step of introducing into the T cell an effective amount of a nucleic acid sequence encoding a forkhead box protein 3 (FOXP3) polypeptide is performed before the step of contacting a T cell with an effective amount of (i) one or more CD3-stimulation agent(s), and (ii) one or more CD28-stimulation agent(s) for a first period of time under conditions that allow for stimulation and activation of the T cell and before the step of contacting the T cell with an effective amount of one or more agent(s) that decreases CD28 expression and/or activity. In some embodiments, contacting the T cell with an effective amount of one or more agent(s) that decreases CD28 expression and/or activity is performed after the step of contacting a T cell with an effective amount of (i) one or more CD3-stimulation agent(s), and (ii) one or more CD28-stimulation agent(s) for a first period of time under conditions that allow for stimulation and activation of the T cell and before the step of introducing into the T cell an effective amount of a nucleic acid sequence encoding a forkhead box protein 3 (FOXP3) polypeptide.

In some embodiments, the method further includes introducing into the T cell an effective amount of a nucleic acid sequence encoding a truncated nerve growth factor receptor (tNGFR) polypeptide.

In some embodiments, the introducing step further includes introducing into a T cell a nucleic acid construct, where the nucleic acid construct includes the nucleic acid sequence encoding the FOXP3 polypeptide.

In some embodiments, the nucleic acid construct, where the nucleic acid further includes a nucleic acid sequence encoding one of the one or more agents that decrease CD28 expression and/or activity. In some embodiments, the one of the one or more agents that decrease CD28 expression and/or activity is a siRNA or a shRNA. In some embodiments, the siRNA includes a sequence of one of SEQ ID NOs: 1-6.

In some embodiments, the nucleic acid construct further includes a promoter operably linked to the nucleic acid sequence encoding the FOXP3 polypeptide.

In some embodiments, the nucleic acid construct further includes a promoter operably linked to the nucleic acid sequence encoding one of the one or more agents that decrease CD28 expression and/or activity.

In some embodiments, the introducing step includes introducing into a T cell a nucleic acid construct, where the nucleic acid construct includes the nucleic acid sequence encoding the FOXP3 polypeptide, a second nucleic acid sequence encoding one of the one or more agents that decrease CD28 expression and/or activity, and a third nucleic acid sequence encoding a tNGFR polypeptide.

In some embodiments, the introducing step further includes introducing into a T cell a nucleic acid construct, where the nucleic acid construct includes the nucleic acid sequence encoding the FOXP3 polypeptide and a second nucleic acid sequence encoding a tNGFR polypeptide. In some embodiments, the introducing step further includes introducing into the T cell a nucleic acid construct including a nucleic acid sequence encoding a tNGFR polypeptide.

In some embodiments, the nucleic acid construct includes a viral vector selected from the group consisting of a lentiviral vector, a retroviral vector, an adenoviral vector, or an adeno-associated viral (AAV) vector. In some embodiments, the viral vector is a lentiviral vector. In some embodiments, the introducing step includes viral transduction.

In some embodiments, the T cell is a CD4⁺ T cell or a CD4⁺/CD45RA⁺ T cell.

In some embodiments, the method further includes, before step (a): obtaining the T cell from a patient or obtaining T cells allogenic to the patient. In some embodiments, the method further includes: treating the obtained T cells to isolate a population of cells enriched for CD4⁺ T cells or CD4⁺/CD45RA⁺ T cells.

In another aspect, this disclosure features a T cell produced by any of the methods described herein.

In another aspect, this disclosure features a composition including any of the T cells described herein.

In another aspect, this disclosure features a T cell including: a first nucleic acid sequence encoding a FOXP3 polypeptide; and one or more agents that decreases CD28 expression and/or activity. In some embodiments, the presence of the first nucleic acid sequence and the one or more agents that decreases CD28 expression and/or activity in the T cell induce the T cell to develop or further develop one or more characteristics of a T regulatory phenotype. In some embodiments, the T cell further includes a third nucleic acid sequence encoding a tNGFR polypeptide. In some embodiments, the one or more agents that decreases CD28 expression and/or activity includes a small interfering RNA (siRNA) or a short hairpin RNA (shRNA). In some embodiments, the siRNA or the shRNA decreases expression of CD28 in a mammalian cell. In some embodiments, the siRNA or shRNA decreases expression of one or more of p85, p110, PIP3, PKB/Akt, mTOR, IκB, GSK3β, NFκB, NFAT, LCK, FYN, and ITK in a T cell. In some embodiments, the siRNA includes a sequence of one of SEQ ID NOs: 1-6.

In another aspect, this disclosure features a T cell including a first nucleic acid sequence encoding a FOXP3 polypeptide; and a second nucleic acid sequence encoding a tNGFR polypeptide. In some embodiments, the presence of the first nucleic acid sequence and the second nucleic acid sequence in the T cell induce the T cell to develop or further develop one or more characteristics of a T regulatory phenotype.

In another aspect, this disclosure features a composition including any of the T cells described herein.

In another aspect, this disclosure features a method of producing a T cell population expressing an exogenous FOXP3 polypeptide and a siRNA where the method includes culturing any of the T cells described herein in growth media under conditions sufficient to expand the population of T cells. In some embodiments, this disclosure includes a population of T cells prepared by the any of the methods described herein.

In another aspect, this disclosure features a vector including a first nucleic acid sequence encoding a FOXP3 polypeptide and a second nucleic acid sequence encoding a siRNA or a shRNA that decreases CD28 expression and/or activity. In some embodiments, the first nucleic acid sequence is operably linked to a promoter, the second nucleic acid sequence is operably linked to a promoter, or the first nucleic acid sequence and the second nucleic acid sequence are both operably linked to a promoter. In some embodiments, the presence of the first nucleic acid sequence and the second nucleic acid sequence in the T cell induce the T cell to develop or further develop one or more characteristics of a T regulatory phenotype.

In another aspect, this disclosure features a vector including a first nucleic acid sequence encoding a FOXP3 polypeptide and a second nucleic acid sequence encoding a tNGFR polypeptide. In some embodiments, the first nucleic acid sequence is operably linked to a promoter, the second nucleic acid sequence is operably linked to a promoter, or the first nucleic acid sequence and the second nucleic acid sequence are both operably linked to a promoter. In some embodiments, the presence of the first nucleic acid sequence and the second nucleic acid sequence in the T cell induce the T cell to develop or further develop one or more characteristics of a T regulatory phenotype.

In another aspect, this disclosure features a vector including first nucleic acid sequence encoding a FOXP3 polypeptide, a second nucleic acid sequence encoding a siRNA or a shRNA that decreases CD28 expression and/or activity, and a third nucleic acid sequence encoding a tNGFR polypeptide. In some embodiments, the first nucleic acid sequence is operably linked to a promoter, the second nucleic acid sequence is operably linked to a promoter, the third nucleic acid sequence is operably linked to a promoter, or the first nucleic acid sequence, the second nucleic acid sequence and the third nucleic acid sequence are all operably linked to a promoter. In some embodiments, the presence of the first nucleic acid sequence, the second nucleic acid sequence, and the third nucleic acid sequence in the T cell induce the T cell to develop or further develop one or more characteristics of a T regulatory phenotype.

In some embodiments, the vector including the siRNA or the shRNA decreases expression of CD28 in a T cell. In some embodiments, the siRNA includes a sequence of one of SEQ ID NOs: 1-6. In some embodiments, the siRNA or shRNA decreases expression of one or more of p85, p110, PIP3, PKB/Akt, mTOR, IκB, GSK3β, NFκB, NFAT, LCK, FYN, and ITK in a T cell.

In some embodiments, the vector includes a viral vector selected from the group consisting of a lentiviral vector, a retroviral vector, an adenoviral vector, or an adeno-associated viral (AAV) vector. In some embodiments, the viral vector is a lentiviral vector.

In another aspect, this disclosure features a composition including any of the vectors described herein.

In another aspect, this disclosure features a kit including the any of the composition described herein.

In another aspect, this disclosure features a method of treating an autoimmune disease or disorder in a patient including administering any one of the T cells described herein or any one of the compositions described herein. In some embodiments, the subject is previously diagnosed or identified as having an autoimmune disease or disorder. In some embodiments, the autoimmune disease or disorder is Lupus, Rheumatoid Arthritis, Multiple Sclerosis, Insulin Dependent Diabetes Mellitis, Myasthenia Gravis, Graves disease, Autoimmune Hemolytic Anemia, Autoimmune Thrombocytopenia Purpura, Goodpasture's Syndrome, Pemphigus Vulgaris, acute Rheumatic Fever, Post-Streptococcal Glomerulonephritis, Crohn's Disease, Celiac Disease, or Polyarteritis Nodosa. In some embodiments, administering the autologous or allogenic T cell population includes intravenous injection or intravenous infusion. In some embodiments, the administering results in amelioration of one or more symptoms of the autoimmune disease or disorder.

Also provided herein are methods of transducing a T cell that include: (a) contacting a T cell with an effective amount of (i) one or more CD3-stimulation agent(s) in the absence of a CD28 stimulating agent for a first period of time under conditions that allow for stimulation and activation of the T cell, and (b) introducing into the T cell an effective amount of a nucleic acid sequence encoding one or more polypeptides operatively linked to a promoter active in T cells, thereby transducing the T cell. Some embodiments of any of the methods described herein further include contacting the T cell with an effective amount of one or more agent(s) that decreases CD28 expression and/or activity.

Also provided herein are methods of transducing a T cell that include: (a) contacting a T cell with an effective amount of (i) one or more CD3-stimulation agent(s), and (ii) one or more CD28-stimulation agent(s) for a first period of time under conditions that allow for stimulation and activation of the T cell; (b) contacting the T cell with an effective amount of one or more agent(s) that decreases CD28 expression and/or activity; and (c) introducing into the T cell an effective amount of a nucleic acid sequence encoding one or more polypeptides operatively linked to a promoter active in T cells, thereby transducing the T cell.

In some embodiments of any of the methods described herein, the one or more CD3-stimulation agent(s) comprises an effective amount of an anti-CD3 antibody. In some embodiments of any of the methods described herein, the one or more CD3-stimulation agent(s) comprise a methyl transferase inhibitor. In some embodiments of any of the methods described herein, the one or more CD28-stimulation agents comprises an anti-CD28 activating antibody. In some embodiments of any of the methods described herein, the one or more agent(s) that decreases CD28 expression and/or activity comprise an anti-CD28 blocking antibody. In some embodiments of any of the methods described herein, the one or more agent(s) that decreases CD28 expression and/or activity comprise a small interfering RNA (siRNA) or a short hairpin RNA (shRNA). In some embodiments of any of the methods described herein, the siRNA or the shRNA decreases expression of CD28 in a T cell. In some embodiments of any of the methods described herein, the siRNA comprises a sequence of one of SEQ ID NOs: 1-6. In some embodiments of any of the methods described herein, the siRNA or shRNA decreases expression of one or more of p85, p110, PIP3, PKB/Akt, mTOR, IκB, GSK3β, NFκB, NFAT, LCK, FYN, and ITK in a T cell. In some embodiments of any of the methods described herein, the method further comprises introducing into the T cell a nucleic acid construct comprising a sequence encoding the siRNA or the shRNA. In some embodiments of any of the methods described herein, the nucleic acid construct further comprises a promoter operably linked to the sequence encoding the shRNA. In some embodiments of any of the methods described herein, the one or more agent(s) that decreases CD28 expression and/or activity comprise a small molecule inhibitor of any one of: LCK, FYN, and ITK.

In some embodiments of any of the methods described herein, the step of contacting of the T cell with an effective amount of one or more agent(s) that decreases CD28 expression and/or activity further comprises removing the one or more agent(s) that decreases CD28 expression and/or activity after about 1 hour to about 60 hours of the first period of time. In some embodiments of any of the methods described herein, step (a) is performed before step (b) and step (c). In some embodiments of any of the methods described herein, step (c) is performed before step (a) and step (b). In some embodiments of any of the methods described herein, step (b) is performed after step (a) and before step (c). In some embodiments of any of the methods described herein, step (a) is performed before step (b). In some embodiments of any of the methods described herein, step (b) is performed before step (a).

In some embodiments of any of the methods described herein, the one or more polypeptides is one more exogenous polypeptides. In some embodiments of any of the methods described herein, one of the one or more polypeptides is a chimeric antigen receptor. In some embodiments of any of the methods described herein, the chimeric antigen receptor comprises an antigen-binding domain capable of binding to CD19.

In some embodiments of any of the methods described herein, the nucleic acid further comprises a nucleic acid sequence encoding one of the one or more agents that decrease CD28 expression and/or activity. In some embodiments of any of the methods described herein, the one of the one or more agents that decreases CD28 expression and/or activity is a siRNA or a shRNA. In some embodiments of any of the methods described herein, the siRNA comprises a sequence of one of SEQ ID NOs: 1-6. In some embodiments of any of the methods described herein, the nucleic acid construct further comprises a promoter that is operably linked to the sequence encoding the siRNA or the shRNA.

In some embodiments of any of the methods described herein, the nucleic acid construct comprises a viral vector selected from the group consisting of a lentiviral vector, a retroviral vector, an adenoviral vector, or an adeno-associated viral (AAV) vector. In some embodiments of any of the methods described herein, the nucleic acid sequence encoding one or more polypeptides operatively linked to the promoter active in T cells is present in a viral vector selected from the group consisting of a lentiviral vector, a retroviral vector, an adenoviral vector, or an adeno-associated viral (AAV) vector. In some embodiments of any of the methods described herein, the viral vector is a lentiviral vector. In some embodiments of any of the methods described herein, the introducing step comprises viral transduction. In some embodiments of any of the methods described herein, the T cell is a CD4⁺ T cell or a CD4⁺/CD45RA⁺ T cell.

Some embodiments of any of the methods described herein further include, before step (a), obtaining the T cell from a patient or obtaining T cells allogenic to the patient. Some embodiments of any of the methods described herein further include treating the obtained T cells to isolate a population of cells enriched for CD4+ T cells or CD4+/CD45RA+ T cells. Also provided herein are T cells produced by any of the methods described herein. Also provided herein are compositions that include any of the T cells described herein and a pharmaceutically acceptable carrier. Also provided herein are methods of treating a subject in need thereof with any of the T cells produced by any of the methods described herein or any of the compositions including any of the T cells produced by any of the methods described herein and a pharmaceutically acceptable carrier.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a schematic of exemplary lentivirus vector constructs. Human FOXP3 and truncated NGFR (tNGFR) or tNGFR alone under the control of an SFFV promoter.

FIG. 2 shows flow cytometry plots measuring FOXP3 and tNGFR four days post-transduction with lentiviruses containing FOXP3+tNGFR or tNGFR alone. Cells were activated with CD3 or CD3/CD28 for 24 hours under different cytokine conditions using ±IL2, ±TGFβ, or a combination of IL2+TGFβ. Cells were transduced with lentiviruses containing FOXP3+tNGFR or tNGFR alone and cultured for 2 days then supplemented with media containing IL2.

FIG. 3 shows the effect of CD3 activation on FOXP3 expression. Mean fluorescence intensity (MFI) of FOXP3⁺ cells in NGFR⁺ population post transduction. MFI at days 4 and 12 post transduction. Top panel represents gating strategy for NGFR⁺ population. Histogram includes the top four rows corresponding to CD3 activated cells; and the bottom four rows corresponding to CD3/CD28 activated cells. IL2 & TGFβ (right) represent treatment conditions during first 3 days post stimulation.

FIG. 4 shows flow cytometry plots measuring FOXP3 and tNGFR in CD3 activated cells. Cells transduced with FOXP3-tNGFR-LV were purified by NGFR columns on day 5 and re-analyzed for FOXP3 expression. Cells were cultured for an additional 7 days as described, and then re-analyzed. Higher fractions of FOXP3⁺ cells were observed in CD3 activation conditions compared to CD3/CD28 activation conditions.

FIG. 5A shows a histogram of fold expansion at day 12 for each condition.

FIG. 5B shows a histogram of FOXP3 expression at day 12 for each condition.

FIG. 5C shows a histogram of CD25 expression at day 12 for each condition.

FIG. 5D shows a histogram of CTLA-4 expression at day 12 for each condition.

FIG. 6A shows a histogram of CD19 CAR expression at day 4 for each condition for donor 1.

FIG. 6B shows a histogram of CD19 CAR expression at day 4 for each condition for donor 2.

### DETAILED DESCRIPTION

Provided herein are methods and materials that can be used to produce T regulatory (Treg) cells that can be used to treat mammals identified as having an autoimmune disease.

In some embodiments, provided herein are methods for producing a T cell by contacting a T cell with an effective amount of (i) one or more CD3 stimulation agent(s) in the absence of a CD28 stimulation agent for a first period of time under conditions that allow for stimulation and activation of the T cell, and introducing into the T cell the T cell an effective amount of a nucleic acid sequence encoding a forkhead box protein 3 (FOXP3) polypeptide, where the presence of the nucleic acid sequence in the T cell induces the T cell to develop or further develop one or more characteristics of a T regulatory cell phenotype compared to when the nucleic acid sequence is not present in the T cell. In some embodiments, the method further comprises contacting the T cell with an effective amount of one or more agent(s) that decreases CD28 expression and/or activity during the first period of time.

In some embodiments, provided herein are methods of producing T regulatory cells by: contacting a T cell with an effective amount of (i) one or more CD3-stimulation agent(s), and (ii) one or more CD28-stimulation agent(s) for a first period of time under conditions that allow for stimulation and activation of the T cell; contacting the T cell with an effective amount of one or more agent(s) that decreases CD28 expression and/or activity; and introducing into the T cell an effective amount of a nucleic acid sequence encoding a forkhead box protein 3 (FOXP3) polypeptide, wherein the presence of the nucleic acid sequence in the T cell induces the T cell to develop or further develop one or more characteristics of a T regulatory cell phenotype compared to when the nucleic acid sequence is not present in the T cell. In some embodiments, the step of contacting the T cell with an effective amount of one or more agent(s) that decreases CD28 expression and/or activity can be performed during the first period of time.

In some embodiments, provided herein are methods for producing a T regulatory cell by contacting a T cell with an effective amount of (i) one or more CD3 stimulation agent(s) and (ii) one or more CD28 stimulation agent(s) for a first period of time under conditions that allow for stimulation and activation of the T cell, and (iii) one or more agent(s) that decreases CD28 expression and/or activity; and introducing into the T cell the T cell an effective amount of a nucleic acid sequence encoding a forkhead box protein 3 (FOXP3) polypeptide, where the presence of the nucleic acid sequence in the T cell induces the T cell to develop or further develop one or more characteristics of a T regulatory cell phenotype compared to when the nucleic acid sequence is not present in the T cell. Also provided herein are methods for producing a T cell by that further includes contacting the T cell with an effective amount of interleuking-2 (IL-2) and/or TGF-β for a second period of time under conditions that allow for stabilization of a T regulatory phenotype as compared to when the T cell is not contacted with IL-2 and/or TGF-β for the second period of time. Also provided herein are methods for treating a mammal having an autoimmune disease, where the method includes administering to the mammal an effective amount of a T cell produced using the methods described herein.

In some embodiments, the method of producing a T regulatory cell includes: contacting a T cell with an effective amount of (i) one or more CD3-stimulation agent(s) for a first period of time under conditions that allow for stimulation and activation of the T cell, and (ii) one or more agent(s) that decreases CD28 expression and/or activity; and introducing into the T cell an effective amount of a nucleic acid sequence encoding a forkhead box protein 3 (FOXP3) polypeptide, wherein the presence of the nucleic acid sequence in the T cell induces the T cell to develop or further develop one or more characteristics of a T regulatory cell phenotype compared to when the nucleic acid sequence is not present in the T cell.

In some embodiments, the method of producing a T regulatory cell includes contacting a T cell with an effective amount of (i) one or more CD3-stimulation agents for a first period of time under conditions that allow for stimulation and activation of the T cell. The CD3-stimulation agents are used to induce robust and reproducible expansion of T cells in vitro. In addition, CD3-stimulation agents can be used to induce activation of T cells in vitro. Following CD3-mediated T cell activation and/or expansion, the T cells can be assessed for transduction capacity (e.g., susceptibility to transduction via, for example, a virus). As described in Example 1, analysis of T cells activated with CD3 and transduced with a lentivirus encoding a FOXP3 polypeptide revealed a surprising increase in transduction efficiency when compared to T cells activated with CD3 and CD28. In some embodiments, the one or more CD3-stimluation agents can include, without limitation, an effective amount of an anti-CD3 antibody and/or a methyl transferase inhibitor. Non-limiting examples of anti-CD3 antibodies includes Biolegend clone UCHT1 and OKT3. Non-limiting examples of a methyl transferase inhibitor include azacytidine, decitabine, and zebularine,

In some embodiments, the method of producing a T regulatory cell includes contacting a T cell with an effective amount of one or more CD28-simulation agent(s) for a first period of time under conditions that allow for stimulation and activation of the T cell. The CD28-stimulation agents are used to induce robust and reproducible expansion of T cells in vitro. The costimulatory receptor CD28 has been shown to be required for the generation of some subsets of Tregs in vivo (Tai, et al., Nat. Immunol., 6:152-162 (2005)). The role of CD28 can be intrinsic to the Tregs (Zhang et al., J. Clin. Invest., 123:580-93 (2013)). A non-limiting example of a CD28 stimulation agent includes an anti-CD28 activating antibody.

In some embodiments where the method of producing a T regulatory cell includes contacting a T cell with an effective amount of (i) one or more CD3-stimulation agent(s), and (ii) one or more CD28-stimulation agent(s) for a first period of time under conditions that allow for stimulation and activation of the T cell, the CD3 and CD28 stimulation agents include beads coated with anti-CD3, anti-CD28, or both anti-CD3 and anti-CD28 antibodies.

In some embodiments, the method of producing a T regulatory cell includes contacting a T cell with an effective amount of one or more agent(s) that decreases CD28 expression and/or activity. In some embodiments, decreasing CD28 expression and/or activity can be used to stabilize T cell stimulation, activation, differentiation, and/or maturation. In some embodiments, decreasing expression and/or activity of CD28 can also be used to stabilize expression of a FOXP3 polypeptide. In some embodiments, decreasing expression and/or activity of CD28 induces the T cell to develop or further develop one or more characteristics of a T regulatory phenotype. Decreasing expression and/or activity of CD28 can also be used to stabilize a T regulatory phenotype in a T cell. Non-limiting examples of an agent that decreases CD28 expression and/or activity of a include: an anti-CD28 blocking antibody, siRNA or shRNA that target CD28 mRNA, siRNA or shRNA that target members of the CD28 signaling pathway (e.g., members of the T cell receptor (TCR) signaling pathway), and/or a small molecule inhibitor of kinases implicated in the CD28 signaling pathway, or any combination thereof. An anti-CD28 blocking antibody can include an antigen-binding fragment (e.g., without limitation, a scFv or a Fab fragment) that binds to a CD28 polypeptide and prevents CD28-mediated signaling (e.g., blocks signaling involving a CD28 polypeptide during T cell activation).

In some embodiments, the one or more agent(s) that decreases CD28 expression and/or activity includes one or more siRNA or one or more shRNA that decrease expression of CD28 in a T cell. Non-limiting examples of siRNA that target CD28 include SEQ ID NO: 1-6 as provided in Table 1.

TABLE 1
siRNA sequences.

| SEQ | | |
|---|---|---|
| ID | | |
| NO: | Identifier | Sequence |
| 1 | CD28 target sequence 1 | CAACCTTAGCTGCAAGTATTC |
| 2 | CD28 target sequence 2 | TGGAGTCCTGGCTTGCTATAG |
| 3 | CD28 target sequence 3 | CCTCCTCCTTACCTAGACAAT |
| 4 | CD28 target sequence 4 | GCTGTGGAAGTCTGTGTTGTA |
| 5 | CD28 target sequence 5 | CGCAAGCATTACCAGCCCTAT |
| 6 | CD28 target sequence 6 | CTTCAATTCAAGTAACAGGAA |

In some embodiments, the method includes introducing into the T cell a nucleic acid construct comprising a nucleic acid sequence encoding the siRNA or the shRNA, where the nucleic acid sequence is operably linked to a promoter. In some embodiments, the nucleic acid sequence encoding a siRNA further comprises one or more additional nucleic acid sequences each encoding an additional siRNA, where each additional nucleic acid sequence comprises a sequence selected from SEQ ID NO: 1-6. In some embodiments, the method includes introducing into the T cell a nucleic acid construct comprising two or more nucleic acid sequence each encoding siRNA or shRNA, where the nucleic acid sequence is operably linked to a promoter.

In some embodiments, the one or more agent(s) that decreases CD28 expression and/or activity include one or more siRNA or one or more shRNA that target an mRNA that directly or indirectly impacts expression of CD28. Non-limiting examples of mRNA that impact expression of CD28 include: p85, p110, PIP3, PKB/Akt, mTOR, IκB, GSK3β, NFκB, NFAT, LCK, FYN, and ITK. In some embodiments, the mRNA that impacts expression of CD28 includes mRNA that are members of the T cell receptor (TCR) signaling pathway.

In some embodiments, the one or more agent(s) that decreases CD28 expression and/or activity includes a small molecule inhibitor of one or more kinases involved in CD28 signaling. Non-limiting examples of kinases that may decrease CD28 expression and/or activity include: LCK, FYN, and ITK.

In some embodiment, a method of producing T regulatory cells includes, for a first period of time, contacting a T cell with an effective amount of (i) one or more CD3-stimulation agent(s) for a first period of time, and (ii) one or more agent(s) that decreases CD28 expression and/or activity under conditions that allow for stimulation and activation of the T cell. As used herein the phrase “a first period of time” includes any period of time sufficient to allow for stimulation and activation of the T cell. For example, a first period of time includes from about 16 hours to about 72 hours (e.g., about 16 hours to about 68 hours, about 16 hours to about 64 hours, about 16 hours to about 60 hours, about 16 hours to about 56 hours, about 16 hours to about 52 hours, about 16 hours to about 48 hours, about 16 hours to about 44 hours, about 16 hours to about 40 hours, about 16 hours to about 36 hours, about 16 hours to about 32 hours, about 16 hours to about 28 hours, about 16 hours to about 24 hours, about 16 hours to about 20 hours, about 20 hours to about 68 hours, about 20 hours to about 60 hours, about 20 hours to about 56 hours, about 20 hours to about 52 hours, about 20 hours to about 48 hours, about 20 hours to about 44 hours, about 20 hours to about 40 hours, about 20 hours to about 36 hours, about 20 hours to about 32 hours, about 20 hours to about 28 hours, about 20 hours to about 24 hours, about 24 hours to about 64 hours, about 24 hours to about 60 hours, about 24 hours to about 56 hours, about 24 hours to about 52, about 24 hours to about 48 hours, about 24 hours to about 44 hours, about 24 hours to about 40 hours, about 24 hours to about 36 hours, about 24 hours to about 32 hours, about 24 hours to about 28 hours, about 28 hours to about 60 hours, about 28 hours to about 56 hours, about 28 hours to about 52 hours, about 28 hours to about 48 hours, about 28 hours to about 44 hours, about 28 hours to about 40 hours, about 28 hours to about 36 hours, about 28 hours to about 32 hours, about 32 hours to about 56 hours, about 32 hours to about 52 hours, about 32 hours to about 48 hours, about 32 hours to about 44 hours, about 32 hours to about 40 hours, about 32 hours to about 36 hours, about 36 hours to about 52 hours, about 36 hours to about 48 hours, about 36 hours to about 44 hours, about 36 hours to about 40 hours, about 40 hours to about 48 hours, about 40 hours to about 44 hours, about 44 hours to about 48 hours, about 48 hours to about 60 hours, about 48 hours to about 72 hours, or about 60 hours to about 72 hours).

In some embodiments where the method includes contacting a T cell during the first period of time with an effective amount of one or more agent(s) that decreases CD28 expression and/or activity, the method includes removing the one or more agent(s) that decreases CD28 expression and/or activity after about 1 hour to about 60 hours (e.g., about 1 hour to about 58 hours, about 1 hour to about 56 hours, about 1 hour to about 54 hours, about 1 hour to about 52 hours, about 1 hour to about 50 hours, about 1 hour to about 48 hours, about 1 hour to about 46 hours, about 1 hour to about 44 hours, about 1 hour to about 42 hours, about 1 hour to about 40 hours, about 1 hour to about 38 hours, about 1 hour to about 36 hours, about 1 hour to about 34 hours, about 1 hour to about 32 hours, about 1 hour to about 30 hours, about 1 hour to about 28 hours, about 1 hour to about 26 hours, about 1 hour to about 24 hours, about 1 hour to about 22 hours, about 1 hour to about 20 hours, about 1 hour to about 18 hours, about 1 hour to about 16 hours, about 1 hour to about 14 hours, about 1 hour to about 12 hours, about 1 hour to about 10 hours, about 1 hour to about 8 hours, about 1 hour to about 6 hours, about 1 hour to about 4 hours, about 1 hour to about 2 hours, about 2 hours to about 32 hours, about 2 hours to about 30 hours, about 2 hours to about 28 hours, about 2 hours to about 26 hours, about 2 hours to about 24 hours, about 2 hours to about 22 hours, about 2 hours to about 20 hours, about 2 hours to about 18 hours, about 2 hours to about 16 hours, about 2 hours to about 14 hours, about 2 hours to about 12 hours, about 2 hours to about 10 hours, about 2 hours to about 8 hours, about 2 hours to about 6 hours, about 2 hours to about 4 hours, about 4 hours to about 32 hours, about 4 hours to about 30 hours, about 4 hours to about 28 hours, about 4 hours to about 26 hours, about 4 hours to about 24 hours, about 4 hours to about 22 hours, about 4 hours to about 20 hours, about 4 hours to about 18 hours, about 4 hours to about 16 hours, about 4 hours to about 14 hours, about 4 hours to about 12 hours, about 4 hours to about 10 hours, about 4 hours to about 8 hours, about 4 hours to about 6 hours, about 6 hours to about 32 hours, about 6 hours to about 30 hours, about 6 hours to about 28 hours, about 6 hours to about 26 hours, about 6 hours to about 24 hours, about 6 hours to about 22 hours, about 6 hours to about 20 hours, about 6 hours to about 18 hours, about 6 hours to about 16 hours, about 6 hours to about 14 hours, about 6 hours to about 12 hours, about 6 hours to about 10 hours, about 6 hours to about 8 hours, about 8 hours to about 32 hours, about 8 hours to about 30 hours, about 8 hours to about 28 hours, about 8 hours to about 26 hours, about 8 hours to about 24 hours, about 8 hours to about 22 hours, about 8 hours to about 20 hours, about 8 hours to about 18 hours, about 8 hours to about 16 hours, about 8 hours to about 14 hours, about 8 hours to about 12 hours, about 8 hours to about 10 hours, about 10 hours to about 32 hours, about 10 hours to about 30 hours, about 10 hours to about 28 hours, about 10 hours to about 26 hours, about 10 hours to about 24 hours, about 10 hours to about 22 hours, about 10 hours to about 20 hours, about 10 hours to about 18 hours, about 10 hours to about 16 hours, about 10 hours to about 14 hours, about 10 hours to about 12 hours, about 12 hours to about 32 hours, about 12 hours to about 30 hours, about 12 hours to about 28 hours, about 12 hours to about 26 hours, about 12 hours to about 24 hours, about 12 hours to about 22 hours, about 12 hours to about 20 hours, about 12 hours to about 18 hours, about 12 hours to about 16 hours, about 12 hours to about 14 hours, about 14 hours to about 32 hours, about 14 hours to about 30 hours, about 14 hours to about 28 hours, about 14 hours to about 26 hours, about 14 hours to about 24 hours, about 14 hours to about 22 hours, about 14 hours to about 20 hours, about 14 hours to about 18 hours, about 14 hours to about 16 hours, about 16 hours to about 32 hours, about 16 hours to about 30 hours, about 16 hours to about 28 hours, about 16 hours to about 26 hours, about 16 hours to about 24 hours, about 16 hours to about 22 hours, about 16 hours to about 20 hours, about 16 hours to about 18 hours, about 18 hours to about 32 hours, about 18 hours to about 30 hours, about 18 hours to about 28 hours, about 18 hours to about 26 hours, about 18 hours to about 24 hours, about 18 hours to about 22 hours, about 18 hours to about 20 hours, about 20 hours to about 32 hours, about 20 hours to about 30 hours, about 20 hours to about 28 hours, about 20 hours to about 26 hours, about 20 hours to about 24 hours, about 20 hours to about 22 hours, about 22 hours to about 32 hours, about 22 hours to about 30 hours, about 22 hours to about 28 hours, about 22 hours to about 26 hours, about 22 hours to about 24 hours, about 24 hours to about 32 hours, about 24 hours to about 30 hours, about 24 hours to about 28 hours, about 24 hours to about 26 hours, about 26 hours to about 32 hours, about 26 hours to about 30 hours, about 26 hours to about 28 hours, about 28 hours to about 32 hours, about 28 hours to about 30 hours, about 30 hours to about 32 hours, about 24 hour to about 50 hours, about 30 hour to about 50 hours, about 36 hour to about 50 hours, about 24 hour to about 48 hours, about 24 hour to about 36 hours, or about 36 hour to about 48 hours) of contact with the T cell.

In some embodiments where the method includes contacting a T cell with an effective amount of one or more agent(s) that decreases CD28 expression and/or activity, the T cell can be contacted with the one or more agent(s) that decreases CD28 expression and/or activity after the first period of time. In such cases, the method can include removing the one or more agent(s) that decreases CD28 expression and/or activity after about 1 hour to about 60 hours (e.g., any of the range limitations described herein) of contact with the T cell.

In some embodiments, the method includes a first period of time where T cells are cultured under conditions that allow for stimulation and activation including first contacting the T cell with an effective amount of one or more agent(s) that decreases CD28 expression and/or activity prior to contacting the T cell with an effective amount of one or more CD3-stimulation agent(s). In some embodiments, the method includes a first period of time where T cells are cultured under conditions that allow for stimulation and activation including first contacting the T cell with an effective amount of one or more CD3-stimulation agent(s) prior to contacting the T cell with an effective amount of one or more agent(s) that decreases CD28 expression and/or activity.

In some embodiments, a method of producing T regulatory cells includes contacting the T cell with an effective amount of interleukin-2 (IL-2) and/or TGF-β for a second period of time under conditions that allow for stabilization of a T regulatory phenotype as compared to when the T cell is not contacted with IL-2 and/or TGF-β for the second period of time. In some embodiments, the method includes not contacting the T cell with IL-2. In some embodiments, the method includes not contacting the T cell with TGF-β. In some embodiments, the method includes not contacting the T cell with either IL-2 or TGF-β. In some embodiments, methods provided herein stabilize a T regulatory phenotype in a T cell, thereby preventing reversion to an effector T cell phenotype.

In some embodiments, a method of producing T regulatory cells includes contacting for a second period of time an effective amount of interleukin-2 (IL-2) and/or TGF-β for a second period of time under conditions that allow for stabilization of a T regulatory cell phenotype as compared to when the T cell is not contacted with IL-2 and/or TGF-β for the second period of time. As used herein “a second period of time” can include any period of time sufficient to induce the T cell to develop or further develop one or more characteristics of a T regulatory phenotype. The second period of time is calculated from day 0 of the method and includes the first period of time. For example, when the method begins on day 0 and the first period of time is about 24 hours (e.g., from day 0 to day 1), the second period of time starts at about day 1. A second period of time includes from about day 1 to about day 14 (e.g., from about day 1 to about day 13, from about day 1 to about day 12, from about day 1 to about day 11, from about day 1 to about day 10, from about day 1 to about day 9, from about day 1 to about day 8, from about day 1 to about day 7, from about day 1 to about day 6, from about day 1 to about day 5, from about day 1 to about day 4, from about day 2 to about day 14, from about day 2 to about day 13, from about day 2 to about day 12, from about day 2 to about day 11, from about day 2 to about day 10, from about day 2 to about day 9, from about day 2 to about day 8, from about day 2 to about day 7, from about day 2 to about day 6, from about day 2 to about day 5, from about day 2 to about day 4, from about day 3 to about day 14, from about day 3 to about day 13, from about day 3 to about day 12, from about day 3 to about day 11, from about day 3 to about day 10, from about day 3 to about day 9, from about day 3 to about day 8, from about day 3 to about day 7, from about day 3 to about day 6, from about day 3 to about day 5, from about day 3 to about day 4, from about day 4 to about day 14, from about day 4 to about day 13, from about day 4 to about day 12, from about day 4 to about day 11, from about day 4 to about day 10, from about day 4 to about day 9, from about day 4 to about day 8, from about day 4 to about day 7, from about day 4 to about day 6, from about day 4 to about day 5, from about day 5 to about day 14, from about day 5 to about day 14, from about day 5 to about day 13, from about day 5 to about day 12, from about day 5 to about day 11, from about day 5 to about day 10, from about day 5 to about day 9, from about day 5 to about day 8, from about day 5 to about day 7, from about day 5 to about day 6, from about day 6 to about day 14, from about day 6 to about day 13, from about day 6 to about day 12, from about day 6 to about day 11, from about day 6 to about day 10, from about day 6 to about day 9, from about day 6 to about day 8, from about day 6 to about day 7, from about day 7 to about day 13, from about day 7 to about day 12, from about day 7 to about day 11, from about day 7 to about day 10, from about day 8 to about day 13, from about day 8 to about day 12, from about day 8 to about day 10, from about day 9 to about day 13, from about day 9 to about day 12, from about day 9 to about day 11, from about day 10 to about day 13, or from about day 11 to about day 13).

In some embodiments, a first period of time is about 24 hours and a second period of time is about 13 days. In a second embodiment, the first period of time is about 48 hours (e.g., day 0 to day 3) and the second period of time is about 12 days.

In some embodiments where the method includes introducing into a T cell an effective amount of a nucleic acid sequence (e.g., a nucleic acid sequence encoding a FOXP3 polypeptide), introducing the nucleic acid sequence into the T cell can occur before, simultaneously with, or after the first period of time.

In some embodiments, the methods include introducing into a T cell an effective amount of a nucleic acid sequence encoding a FOXP3 polypeptide. In some embodiments, the presence of a nucleic acid sequence encoding a FOXP3 polypeptide in a T cell (e.g., any of the T cells described herein) elicits a Treg phenoytpe in the T cell as compared to when the FOXP3 polypeptide is not present in the mammalian cell.

As used herein, “FOXP3” refers to the FOXP3 gene that is a transcription factor in the Forkhead box (Fox) family of transcription factors (Sakaguchi et al., Int'l Immun., 21(10):1105-1111 (2009); Pandiyan, et al., Cytokine, 76(1):13-24 (2015)). In some embodiments, when preparing a T cell to be used in the treatment of a mammal having an autoimmune disease by administering to the mammal the T cell, FOXP3 refers to human FOXP3. An example of a human FOXP3 polypeptide includes, without limitation, NCBI reference sequence: NP 001107849.1 or a fragment thereof.

In some embodiments referring to a nucleic acid sequence encoding a FOXP3 (e.g., full length FOXP3) polypeptide, the nucleic acid sequence is at least 70% (e.g., at least 75%, 80%, 85%, 90%, 95%, 99% and 100%) identical to SEQ ID NO: 7:

AGTTTCCCACAAGCCAGGCTGATCCTTTTCTGTCAGTCCACTTCACCAA
GCCTGCCCTTGGACAAGGACCCGATGCCCAACCCCAGGCCTGGCAAGCC
CTCGGCCCCTTCCTTGGCCCTTGGCCCATCCCCAGGAGCCTCGCCCAGC
TGGAGGGCTGCACCCAAAGCCTCAGACCTGCTGGGGGCCCGGGGCCCAG
GGGGAACCTTCCAGGGCCGAGATCTTCGAGGCGGGGCCCATGCCTCCTC
TTCTTCCTTGAACCCCATGCCACCATCGCAGCTGCAGCTGCCCACACTG
CCCCTAGTCATGGTGGCACCCTCCGGGGCACGGCTGGGCCCCTTGCCCC
ACTTACAGGCACTCCTCCAGGACAGGCCACATTTCATGCACCAGCTCTC
AACGGTGGATGCCCACGCCCGGACCCCTGTGCTGCAGGTGCACCCCCTG
GAGAGCCCAGCCATGATCAGCCTCACACCACCCACCACCGCCACTGGGG
TCTTCTCCCTCAAGGCCCGGCCTGGCCTCCCACCTGGGATCAACGTGGC
CAGCCTGGAATGGGTGTCCAGGGAGCCGGCACTGCTCTGCACCTTCCCA
AATCCCAGTGCACCCAGGAAGGACAGCACCCTTTCGGCTGTGCCCCAGA
GCTCCTACCCACTGCTGGCAAATGGTGTCTGCAAGTGGCCCGGATGTGA
GAAGGTCTTCGAAGAGCCAGAGGACTTCCTCAAGCACTGCCAGGCGGAC
CATCTTCTGGATGAGAAGGGCAGGGCACAATGTCTCCTCCAGAGAGAGA
TGGTACAGTCTCTGGAGCAGCAGCTGGTGCTGGAGAAGGAGAAGCTGAG
TGCCATGCAGGCCCACCTGGCTGGGAAAATGGCACTGACCAAGGCTTCA
TCTGTGGCATCATCCGACAAGGGCTCCTGCTGCATCGTAGCTGCTGGCA
GCCAAGGCCCTGTCGTCCCAGCCTGGTCTGGCCCCCGGGAGGCCCCTGA
CAGCCTGTTTGCTGTCCGGAGGCACCTGTGGGGTAGCCATGGAAACAGC
ACATTCCCAGAGTTCCTCCACAACATGGACTACTTCAAGTTCCACAACA
TGCGACCCCCTTTCACCTACGCCACGCTCATCCGCTGGGCCATCCTGGA
GGCTCCAGAGAAGCAGCGGACACTCAATGAGATCTACCACTGGTTCACA
CGCATGTTTGCCTTCTTCAGAAACCATCCTGCCACCTGGAAGAACGCCA
TCCGCCACAACCTGAGTCTGCACAAGTGCTTTGTGCGGGTGGAGAGCGA
GAAGGGGGCTGTGTGGACCGTGGATGAGCTGGAGTTCCGCAAGAAACGG
AGCCAGAGGCCCAGCAGGTGTTCCAACCCTACACCTGGCCCCTGACCTC
AAGATCAAGGAAAGGAGGATGGACGAACAGGGGCCAAACTGGTGGGAGG
CAGAGGTGGTGGGGGCAGGGATGATAGGCCCTGGATGTGCCCACAGGGA
CCAAGAAGTGAGGTTTCCACTGTCTTGCCTGCCAGGGCCCCTGTTCCCC
CGCTGGCAGCCACCCCCTCCCCCATCATATCCTTTGCCCCAAGGCTGCT
CAGAGGGGCCCCGGTCCTGGCCCCAGCCCCCACCTCCGCCCCAGACACA
CCCCCCAGTCGAGCCCTGCAGCCAAACAGAGCCTTCACAACCAGCCACA
CAGAGCCTGCCTCAGCTGCTCGCACAGATTACTTCAGGGCTGGAAAAGT
CACACAGACACACAAAATGTCACAATCCTGTCCCTCACTCAACACAAAC
CCCAAAACACAGAGAGCCTGCCTCAGTACACTCAAACAACCTCAAAGCT
GCATCATCACACAATCACACACAAGCACAGCCCTGACAACCCACACACC
CCAAGGCACGCACCCACAGCCAGCCTCAGGGCCCACAGGGGCACTGTCA
ACACAGGGGTGTGCCCAGAGGCCTACACAGAAGCAGCGTCAGTACCCTC
AGGATCTGAGGTCCCAACACGTGCTCGCTCACACACACGGCCTGTTAGA
ATTCACCTGTGTATCTCACGCATATGCACACGCACAGCCCCCCAGTGGG
TCTCTTGAGTCCCGTGCAGACACACACAGCCACACACACTGCCTTGCCA
AAAATACCCCGTGTCTCCCCTGCCACTCACCTCACTCCCATTCCCTGAG
CCCTGATCCATGCCTCAGCTTAGACTGCAGAGGAACTACTCATTTATTT
GGGATCCAAGGCCCCCAACCCACAGTACCGTCCCCAATAAACTGCAGCC
GAGCTCCCCA.

In some embodiments referring to a nucleic acid sequence encoding a FOXP3 (e.g., full length FOXP3) polypeptide, the nucleic acid sequence is a codon optimized version of SEQ ID NO: 7. For example, the codon optimized version of the nucleic acid sequence encoding FOXP3 includes one or more nucleotide differences in the nucleic acid sequence of SEQ ID NO: 7 and still encodes for the same amino acid sequence that is encoded in the nucleic acid sequence of SEQ ID NO: 7.

In some embodiments, the amino acid sequence of a FOXP3 polypeptide is at least 80% (e.g., at least 85%, at least 90%, at least 95%, at least 98% or at least 99%) identical to SEQ ID NO: 8:

MPNPRPGKPSAPSLALGPSPGASPSWRAAPKASDLLGARGPGGTFQGRD
LRGGAHASSSSLNPMPPSQLQLPTLPLVMVAPSGARLGPLPHLQALLQD
RPHFMHQLSTVDAHARTPVLQVHPLESPAMISLTPPTTATGVFSLKARP
GLPPGINVASLEWVSREPALLCTFPNPSAPRKDSTLSAVPQSSYPLLAN
GVCKWPGCEKVFEEPEDFLKHCQADHLLDEKGRAQCLLQREMVQSLEQQ
LVLEKEKLSAMQAHLAGKMALTKASSVASSDKGSCCIVAAGSQGPVVPA
WSGPREAPDSLFAVRRHLWGSHGNSTFPEFLHNMDYFKFHNMRPPFTYA
TLIRWAILEAPEKQRTLNEIYHWFTRMFAFFRNHPATWKNAIRHNLSLH
KCFVRVESEKGAVWTVDELEFRKKRSQRPSRCSNPTPGP.

In some embodiments, the method further includes introducing into a T cell (e.g., a CD4⁺ T cell, a CD4⁺ CD45RA⁺ T cell, a CD4⁺ CD62L⁺ T cell, or a central memory T cell) an effective amount of a sequence encoding a truncated nerve growth factor receptor (tNGFR) polypeptide. NGFR (nerve growth factor receptor), also known as Low affinity neurotrophin receptor p75NTR and CD271, is a single-pass type I transmembrane glycoprotein in the TNF receptor superfamily (TNFRSF16). NGFR or truncated NGFR (tNGFR) can be used as a marker for transduction efficiency. In some embodiments, the nucleic acid sequence encoding a wildtype tNGFR polypeptide includes a sequence that is at least 70% (e.g., at least 75%, 80%, 85%, 90%, 95%, 99% and 100%) identical to SE ID NO: 9:

ATGGCCACAACCATGGACGGGCCGCGCCTGCTGCTGTTGCTGCTTCTGG
GGGTGTCCCTTGGAGGTGCCAAGGAGGCATGCCCCACAGGCCTGTACAC
ACACAGCGGTGAGTGCTGCAAAGCCTGCAACCTGGGCGAGGGTGTGGCC
CAGCCTTGTGGAGCCAACCAGACCGTGTGTGAGCCCTGCCTGGACAGCG
TGACGTTCTCCGACGTGGTGAGCGCGACCGAGCCGTGCAAGCCGTGCAC
CGAGTGCGTGGGGCTCCAGAGCATGTCGGCGCCGTGCGTGGAGGCCGAC
GACGCCGTGTGCCGCTGCGCCTACGGCTACTACCAGGATGAGACGACTG
GGCGCTGCGAGGCGTGCCGCGTGTGCGAGGCGGGCTCGGGCCTCGTGTT
CTCCTGCCAGGACAAGCAGAACACCGTGTGCGAGGAGTGCCCCGACGGC
ACGTATTCCGACGAGGCCAACCACGTGGACCCGTGCCTGCCCTGCACCG
TGTGCGAGGACACCGAGCGCCAGCTCCGCGAGTGCACACGCTGGGCCGA
CGCCGAGTGCGAGGAGATCCCTGGCCGTTGGATTACACGGTCCACACCC
CCAGAGGGCTCGGACAGCACAGCCCCCAGCACCCAGGAGCCTGAGGCAC
CTCCAGAACAAGACCTCATAGCCAGCACGGTGGCAGGTGTGGTGACCAC
AGTGATGGGCAGCTCCCAGCCCGTGGTGACCCGAGGCACCACCGACAAC
CTCATCCCTGTCTATTGCTCCATCCTGGCTGCTGTGGTTGTGGGCCTTG
TGGCCTACATAGCCTTCAAGAGGTGGAACAGTCATCGATATCCTCGAGG
TCACCGCGGTCTAGAGTCGACCTGCAGCCAAGCTTATCGATAA

In some embodiments, the amino acid sequence of a tNGFR polypeptide is at least 80% (e.g., at least 85%, at least 90%, at least 95%, at least 98% or at least 99%) identical to SEQ ID NO: 10:

MGAGATGRAMDGPRLLLLLLLGVSLGGAKEACPTGLYTHSGECCKACNL
GEGVAQPCGANQTVCEPCLDSVTFSDVVSATEPCKPCTECVGLQSMSAP
CVEADDAVCRCAYGYYQDETTGRCEACRVCEAGSGLVFSCQDKQNTVCE
ECPDGTYSDEANHVDPCLPCTVCEDTERQLRECTRWADAECEEIPGRWI
TRSTPPEGSDSTAPSTQEPEAPPEQDLIASTVAGVVTTVMGSSQPVVTR
GTTDNLIPVYCSILAAVVVGLVAYIAFKRWNS.

CD28, also known as tp44, is essential for T cell proliferation and survival, cytokine production, among other cellular processes. An example of a human CD28 polypeptide includes, without limitation, NCBI reference sequence: NP_001230006.1 or a fragment thereof, NP_001230007.1 or a fragment thereof, or NP_006130.1 or a fragment thereof. In some embodiments, the amino acid sequence of a CD28 polypeptide is at least 80% (e.g., at least 85%, at least 90%, at least 95%, at least 98% or at least 99%) identical to SEQ ID NO: 11:

MLRLLLALNLFPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS.

In some embodiments, the nucleic acid sequence encoding a wild type CD28 polypeptide includes a sequence that is at least 70% (e.g., at least 75%, 80%, 85%, 90%, 95%, 99% and 100%) identical to SEQ ID NO: 12:

ACACTTCGGGTTCCTCGGGGAGGAGGGGCTGGAACCCTAGCCCATCGTCAGGACAA
AGATGCTCAGGCTGCTCTTGGCTCTCAACTTATTCCCTTCAATTCAAGTAACAGGAA
ACAAGATTTTGGTGAAGCAGTCGCCCATGCTTGTAGCGTACGACAATGCGGTCAACC
TTAGCTGGAAACACCTTTGTCCAAGTCCCCTATTTCCCGGACCTTCTAAGCCCTTTTG
GGTGCTGGTGGTGGTTGGTGGAGTCCTGGCTTGCTATAGCTTGCTAGTAACAGTGGC
CTTTATTATTTTCTGGGTGAGGAGTAAGAGGAGCAGGCTCCTGCACAGTGACTACAT
GAACATGACTCCCCGCCGCCCCGGGCCCACCCGCAAGCATTACCAGCCCTATGCCCC
ACCACGCGACTTCGCAGCCTATCGCTCCTGACACGGACGCCTATCCAGAAGCCAGCC
GGCTGGCAGCCCCCATCTGCTCAATATCACTGCTCTGGATAGGAAATGACCGCCATC
TCCAGCCGGCCACCTCAGGCCCCTGTTGGGCCACCAATGCCAATTTTTCTCGAGTGA
CTAGACCAAATATCAAGATCATTTTGAGACTCTGAAATGAAGTAAAAGAGATTTCCT
GTGACAGGCCAAGTCTTACAGTGCCATGGCCCACATTCCAACTTACCATGTACTTAG
TGACTTGACTGAGAAGTTAGGGTAGAAAACAAAAAGGGAGTGGATTCTGGGAGCCT
CTTCCCTTTCTCACTCACCTGCACATCTCAGTCAAGCAAAGTGTGGTATCCACAGAC
ATTTTAGTTGCAGAAGAAAGGCTAGGAAATCATTCCTTTTGGTTAAATGGGTGTTTA
ATCTTTTGGTTAGTGGGTTAAACGGGGTAAGTTAGAGTAGGGGGAGGGATAGGAAG
ACATATTTAAAAACCATTAAAACACTGTCTCCCACTCATGAAATGAGCCACGTAGTT
CCTATTTAATGCTGTTTTCCTTTAGTTTAGAAATACATAGACATTGTCTTTTATGAAT
TCTGATCATATTTAGTCATTTTGACCAAATGAGGGATTTGGTCAAATGAGGGATTCC
CTCAAAGCAATATCAGGTAAACCAAGTTGCTTTCCTCACTCCCTGTCATGAGACTTC
AGTGTTAATGTTCACAATATACTTTCGAAAGAATAAAATAGTTCTCCTACATGAAGA
AAGAATATGTCAGGAAATAAGGTCACTTTATGTCAAAATTATTTGAGTACTATGGGA
CCTGGCGCAGTGGCTCATGCTTGTAATCCCAGCACTTTGGGAGGCCGAGGTGGGCAG
ATCACTTGAGATCAGGACCAGCCTGGTCAAGATGGTGAAACTCCGTCTGTACTAAAA
ATACAAAATTTAGCTTGGCCTGGTGGCAGGCACCTGTAATCCCAGCTGCCCAAGAGG
CTGAGGCATGAGAATCGCTTGAACCTGGCAGGCGGAGGTTGCAGTGAGCCGAGATA
GTGCCACAGCTCTCCAGCCTGGGCGACAGAGTGAGACTCCATCTCAAACAACAACA
ACAACAACAACAACAACAACAAACCACAAAATTATTTGAGTACTGTGAAGGATTAT
TTGTCTAACAGTTCATTCCAATCAGACCAGGTAGGAGCTTTCCTGTTTCATATGTTTC
AGGGTTGCACAGTTGGTCTCTTTAATGTCGGTGTGGAGATCCAAAGTGGGTTGTGGA
AAGAGCGTCCATAGGAGAAGTGAGAATACTGTGAAAAAGGGATGTTAGCATTCATT
AGAGTATGAGGATGAGTCCCAAGAAGGTTCTTTGGAAGGAGGACGAATAGAATGGA
GTAATGAAATTCTTGCCATGTGCTGAGGAGATAGCCAGCATTAGGTGACAATCTTCC
AGAAGTGGTCAGGCAGAAGGTGCCCTGGTGAGAGCTCCTTTACAGGGACTTTATGT
GGTTTAGGGCTCAGAGCTCCAAAACTCTGGGCTCAGCTGCTCCTGTACCTTGGAGGT
CCATTCACATGGGAAAGTATTTTGGAATGTGTCTTTTGAAGAGAGCATCAGAGTTCT
TAAGGGACTGGGTAAGGCCTGACCCTGAAATGACCATGGATATTTTTCTACCTACAG
TTTGAGTCAACTAGAATATGCCTGGGGACCTTGAAGAATGGCCCTTCAGTGGCCCTC
ACCATTTGTTCATGCTTCAGTTAATTCAGGTGTTGAAGGAGCTTAGGTTTTAGAGGC
ACGTAGACTTGGTTCAAGTCTCGTTAGTAGTTGAATAGCCTCAGGCAAGTCACTGCC
CACCTAAGATGATGGTTCTTCAACTATAAAATGGAGATAATGGTTACAAATGTCTCT
TCCTATAGTATAATCTCCATAAGGGCATGGCCCAAGTCTGTCTTTGACTCTGCCTATC
CCTGACATTTAGTAGCATGCCCGACATACAATGTTAGCTATTGGTATTATTGCCATAT
AGATAAATTATGTATAAAAATTAAACTGGGCAATAGCCTAAGAAGGGGGGAATATT
GTAACACAAATTTAAACCCACTACGCAGGGATGAGGTGCTATAATATGAGGACCTTT
TAACTTCCATCATTTTCCTGTTTCTTGAAATAGTTTATCTTGTAATGAAATATAAGGC
ACCTCCCACTTTTATGTATAGAAAGAGGTCTTTTAATTTTTTTTTAATGTGAGAAGGA
AGGGAGGAGTAGGAATCTTGAGATTCCAGATCGAAAATACTGTACTTTGGTTGATTT
TTAAGTGGGCTTCCATTCCATGGATTTAATCAGTCCCAAGAAGATCAAACTCAGCAG
TACTTGGGTGCTGAAGAACTGTTGGATTTACCCTGGCACGTGTGCCACTTGCCAGCT
TCTTGGGCACACAGAGTTCTTCAATCCAAGTTATCAGATTGTATTTGAAAATGACAG
AGCTGGAGAGTTTTTTGAAATGGCAGTGGCAAATAAATAAATACTTTTTTTTAAATG
GAAAGACTTGATCTATGGTAATAAATGATTTTGTTTTCTGACTGGAAAAATAGGCCT
ACTAAAGATGAATCACACTTGAGATGTTTCTTACTCACTCTGCACAGAAACAAAGAA
GAAATGTTATACAGGGAAGTCCGTTTTCACTATTAGTATGAACCAAGAAATGGTTCA
AAAACAGTGGTAGGAGCAATGCTTTCATAGTTTCAGATATGGTAGTTATGAAGAAA
ACAATGTCATTTGCTGCTATTATTGTAAGAGTCTTATAATTAATGGTACTCCTATAAT
TTTTGATTGTGAGCTCACCTATTTGGGTTAAGCATGCCAATTTAAAGAGACCAAGTG
TATGTACATTATGTTCTACATATTCAGTGATAAAATTACTAAACTACTATATGTCTGC
TTTAAATTTGTACTTTAATATTGTCTTTTGGTATTAAGAAAGATATGCTTTCAGAATA
GATATGCTTCGCTTTGGCAAGGAATTTGGATAGAACTTGCTATTTAAAAGAGGTGTG
GGGTAAATCCTTGTATAAATCTCCAGTTTAGCCTTTTTTGAAAAAGCTAGACTTTCAA
ATACTAATTTCACTTCAAGCAGGGTACGTTTCTGGTTTGTTTGCTTGACTTCAGTCAC
AATTTCTTATCAGACCAATGGCTGACCTCTTTGAGATGTCAGGCTAGGCTTACCTAT
GTGTTCTGTGTCATGTGAATGCTGAGAAGTTTGACAGAGATCCAACTTCAGCCTTGA
CCCCATCAGTCCCTCGGGTTAACTAACTGAGCCACCGGTCCTCATGGCTATTTTAAT
GAGGGTATTGATGGTTAAATGCATGTCTGATCCCTTATCCCAGCCATTTGCACTGCC
AGCTGGGAACTATACCAGACCTGGATACTGATCCCAAAGTGTTAAATTCAACTACAT
GCTGGAGATTAGAGATGGTGCCAATAAAGGACCCAGAACCAGGATCTTGATTGCTA
TAGACTTATTAATAATCCAGGTCAAAGAGAGTGACACACACTCTCTCAAGACCTGGG
GTGAGGGAGTCTGTGTTATCTGCAAGGCCATTTGAGGCTCAGAAAGTCTCTCTTTCC
TATAGATATATGCATACTTTCTGACATATAGGAATGTATCAGGAATACTCAACCATC
ACAGGCATGTTCCTACCTCAGGGCCTTTACATGTCCTGTTTACTCTGTCTAGAATGTC
CTTCTGTAGATGACCTGGCTTGCCTCGTCACCCTTCAGGTCCTTGCTCAAGTGTCATC
TTCTCCCCTAGTTAAACTACCCCACACCCTGTCTGCTTTCCTTGCTTATTTTTCTCCAT
AGCATTTTACCATCTCTTACATTAGACATTTTTCTTATTTATTTGTAGTTTATAAGCTT
CATGAGGCAAGTAACTTTGCTTTGTTTCTTGCTGTATCTCCAGTGCCCAGAGCAGTGC
CTGGTATATAATAAATATTTATTGACTGAGTGAA.

In some embodiments, the nucleic acid sequence encoding a wild type CD28 polypeptide includes a sequence that is at least 70% (e.g., at least 75%, 80%, 85%, 90%, 95%, 99% and 100%) identical to SEQ ID NO: 13:

TAAAGTCATCAAAACAACGTTATATCCTGTGTGAAATGCTGCAGTCAGGATGCCTTG
TGGTTTGAGTGCCTTGATCATGTGCCCTAAGGGGATGGTGGCGGTGGTGGTGGCCGT
GGATGACGGAGACTCTCAGGCCTTGGCAGGTGCGTCTTTCAGTTCCCCTCACACTTC
GGGTTCCTCGGGGAGGAGGGGCTGGAACCCTAGCCCATCGTCAGGACAAAGATGCT
CAGGCTGCTCTTGGCTCTCAACTTATTCCCTTCAATTCAAGTAACAGGGAAACACCT
TTGTCCAAGTCCCCTATTTCCCGGACCTTCTAAGCCCTTTTGGGTGCTGGTGGTGGTT
GGTGGAGTCCTGGCTTGCTATAGCTTGCTAGTAACAGTGGCCTTTATTATTTTCTGGG
TGAGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCGCA
GCCTATCGCTCCTGACACGGACGCCTATCCAGAAGCCAGCCGGCTGGCAGCCCCCAT
CTGCTCAATATCACTGCTCTGGATAGGAAATGACCGCCATCTCCAGCCGGCCACCTC
AGGCCCCTGTTGGGCCACCAATGCCAATTTTTCTCGAGTGACTAGACCAAATATCAA
GATCATTTTGAGACTCTGAAATGAAGTAAAAGAGATTTCCTGTGACAGGCCAAGTCT
TACAGTGCCATGGCCCACATTCCAACTTACCATGTACTTAGTGACTTGACTGAGAAG
TTAGGGTAGAAAACAAAAAGGGAGTGGATTCTGGGAGCCTCTTCCCTTTCTCACTCA
CCTGCACATCTCAGTCAAGCAAAGTGTGGTATCCACAGACATTTTAGTTGCAGAAGA
AAGGCTAGGAAATCATTCCTTTTGGTTAAATGGGTGTTTAATCTTTTGGTTAGTGGGT
TAAACGGGGTAAGTTAGAGTAGGGGGAGGGATAGGAAGACATATTTAAAAACCATT
AAAACACTGTCTCCCACTCATGAAATGAGCCACGTAGTTCCTATTTAATGCTGTTTTC
CTTTAGTTTAGAAATACATAGACATTGTCTTTTATGAATTCTGATCATATTTAGTCAT
TTTGACCAAATGAGGGATTTGGTCAAATGAGGGATTCCCTCAAAGCAATATCAGGTA
AACCAAGTTGCTTTCCTCACTCCCTGTCATGAGACTTCAGTGTTAATGTTCACAATAT
ACTTTCGAAAGAATAAAATAGTTCTCCTACATGAAGAAAGAATATGTCAGGAAATA
AGGTCACTTTATGTCAAAATTATTTGAGTACTATGGGACCTGGCGCAGTGGCTCATG
CTTGTAATCCCAGCACTTTGGGAGGCCGAGGTGGGCAGATCACTTGAGATCAGGAC
CAGCCTGGTCAAGATGGTGAAACTCCGTCTGTACTAAAAATACAAAATTTAGCTTGG
CCTGGTGGCAGGCACCTGTAATCCCAGCTGCCCAAGAGGCTGAGGCATGAGAATCG
CTTGAACCTGGCAGGCGGAGGTTGCAGTGAGCCGAGATAGTGCCACAGCTCTCCAG
CCTGGGCGACAGAGTGAGACTCCATCTCAAACAACAACAACAACAACAACAACAAC
AACAAACCACAAAATTATTTGAGTACTGTGAAGGATTATTTGTCTAACAGTTCATTC
CAATCAGACCAGGTAGGAGCTTTCCTGTTTCATATGTTTCAGGGTTGCACAGTTGGT
CTCTTTAATGTCGGTGTGGAGATCCAAAGTGGGTTGTGGAAAGAGCGTCCATAGGA
GAAGTGAGAATACTGTGAAAAAGGGATGTTAGCATTCATTAGAGTATGAGGATGAG
TCCCAAGAAGGTTCTTTGGAAGGAGGACGAATAGAATGGAGTAATGAAATTCTTGC
CATGTGCTGAGGAGATAGCCAGCATTAGGTGACAATCTTCCAGAAGTGGTCAGGCA
GAAGGTGCCCTGGTGAGAGCTCCTTTACAGGGACTTTATGTGGTTTAGGGCTCAGAG
CTCCAAAACTCTGGGCTCAGCTGCTCCTGTACCTTGGAGGTCCATTCACATGGGAAA
GTATTTTGGAATGTGTCTTTTGAAGAGAGCATCAGAGTTCTTAAGGGACTGGGTAAG
GCCTGACCCTGAAATGACCATGGATATTTTTCTACCTACAGTTTGAGTCAACTAGAA
TATGCCTGGGGACCTTGAAGAATGGCCCTTCAGTGGCCCTCACCATTTGTTCATGCTT
CAGTTAATTCAGGTGTTGAAGGAGCTTAGGTTTTAGAGGCACGTAGACTTGGTTCAA
GTCTCGTTAGTAGTTGAATAGCCTCAGGCAAGTCACTGCCCACCTAAGATGATGGTT
CTTCAACTATAAAATGGAGATAATGGTTACAAATGTCTCTTCCTATAGTATAATCTC
CATAAGGGCATGGCCCAAGTCTGTCTTTGACTCTGCCTATCCCTGACATTTAGTAGC
ATGCCCGACATACAATGTTAGCTATTGGTATTATTGCCATATAGATAAATTATGTAT
AAAAATTAAACTGGGCAATAGCCTAAGAAGGGGGGAATATTGTAACACAAATTTAA
ACCCACTACGCAGGGATGAGGTGCTATAATATGAGGACCTTTTAACTTCCATCATTT
TCCTGTTTCTTGAAATAGTTTATCTTGTAATGAAATATAAGGCACCTCCCACTTTTAT
GTATAGAAAGAGGTCTTTTAATTTTTTTTTAATGTGAGAAGGAAGGGAGGAGTAGGA
ATCTTGAGATTCCAGATCGAAAATACTGTACTTTGGTTGATTTTTAAGTGGGCTTCCA
TTCCATGGATTTAATCAGTCCCAAGAAGATCAAACTCAGCAGTACTTGGGTGCTGAA
GAACTGTTGGATTTACCCTGGCACGTGTGCCACTTGCCAGCTTCTTGGGCACACAGA
GTTCTTCAATCCAAGTTATCAGATTGTATTTGAAAATGACAGAGCTGGAGAGTTTTT
TGAAATGGCAGTGGCAAATAAATAAATACTTTTTTTTAAATGGAAAGACTTGATCTA
TGGTAATAAATGATTTTGTTTTCTGACTGGAAAAATAGGCCTACTAAAGATGAATCA
CACTTGAGATGTTTCTTACTCACTCTGCACAGAAACAAAGAAGAAATGTTATACAGG
GAAGTCCGTTTTCACTATTAGTATGAACCAAGAAATGGTTCAAAAACAGTGGTAGG
AGCAATGCTTTCATAGTTTCAGATATGGTAGTTATGAAGAAAACAATGTCATTTGCT
GCTATTATTGTAAGAGTCTTATAATTAATGGTACTCCTATAATTTTTGATTGTGAGCT
CACCTATTTGGGTTAAGCATGCCAATTTAAAGAGACCAAGTGTATGTACATTATGTT
CTACATATTCAGTGATAAAATTACTAAACTACTATATGTCTGCTTTAAATTTGTACTT
TAATATTGTCTTTTGGTATTAAGAAAGATATGCTTTCAGAATAGATATGCTTCGCTTT
GGCAAGGAATTTGGATAGAACTTGCTATTTAAAAGAGGTGTGGGGTAAATCCTTGTA
TAAATCTCCAGTTTAGCCTTTTTTGAAAAAGCTAGACTTTCAAATACTAATTTCACTT
CAAGCAGGGTACGTTTCTGGTTTGTTTGCTTGACTTCAGTCACAATTTCTTATCAGAC
CAATGGCTGACCTCTTTGAGATGTCAGGCTAGGCTTACCTATGTGTTCTGTGTCATGT
GAATGCTGAGAAGTTTGACAGAGATCCAACTTCAGCCTTGACCCCATCAGTCCCTCG
GGTTAACTAACTGAGCCACCGGTCCTCATGGCTATTTTAATGAGGGTATTGATGGTT
AAATGCATGTCTGATCCCTTATCCCAGCCATTTGCACTGCCAGCTGGGAACTATACC
AGACCTGGATACTGATCCCAAAGTGTTAAATTCAACTACATGCTGGAGATTAGAGAT
GGTGCCAATAAAGGACCCAGAACCAGGATCTTGATTGCTATAGACTTATTAATAATC
CAGGTCAAAGAGAGTGACACACACTCTCTCAAGACCTGGGGTGAGGGAGTCTGTGT
TATCTGCAAGGCCATTTGAGGCTCAGAAAGTCTCTCTTTCCTATAGATATATGCATA
CTTTCTGACATATAGGAATGTATCAGGAATACTCAACCATCACAGGCATGTTCCTAC
CTCAGGGCCTTTACATGTCCTGTTTACTCTGTCTAGAATGTCCTTCTGTAGATGACCT
GGCTTGCCTCGTCACCCTTCAGGTCCTTGCTCAAGTGTCATCTTCTCCCCTAGTTAAA
CTACCCCACACCCTGTCTGCTTTCCTTGCTTATTTTTCTCCATAGCATTTTACCATCTC
TTACATTAGACATTTTTCTTATTTATTTGTAGTTTATAAGCTTCATGAGGCAAGTAAC
TTTGCTTTGTTTCTTGCTGTATCTCCAGTGCCCAGAGCAGTGCCTGGTATATAATAAA
TATTTATTGACTGAGTGAAAAAAAAAAAAAAAAA.

In some embodiments, the nucleic acid sequence encoding a wild type CD28 polypeptide includes a sequence that is at least 70% (e.g., at least 75%, 80%, 85%, 90%, 95%, 99% and 100%) identical to SEQ ID NO: 14:

ACACTTCGGGTTCCTCGGGGAGGAGGGGCTGGAACCCTAGCCCATCGTCAGGACAA
AGATGCTCAGGCTGCTCTTGGCTCTCAACTTATTCCCTTCAATTCAAGTAACAGGAA
ACAAGATTTTGGTGAAGCAGTCGCCCATGCTTGTAGCGTACGACAATGCGGTCAACC
TTAGCTGCAAGTATTCCTACAATCTCTTCTCAAGGGAGTTCCGGGCATCCCTTCACA
AAGGACTGGATAGTGCTGTGGAAGTCTGTGTTGTATATGGGAATTACTCCCAGCAGC
TTCAGGTTTACTCAAAAACGGGGTTCAACTGTGATGGGAAATTGGGCAATGAATCA
GTGACATTCTACCTCCAGAATTTGTATGTTAACCAAACAGATATTTACTTCTGCAAA
ATTGAAGTTATGTATCCTCCTCCTTACCTAGACAATGAGAAGAGCAATGGAACCATT
ATCCATGTGAAAGGGAAACACCTTTGTCCAAGTCCCCTATTTCCCGGACCTTCTAAG
CCCTTTTGGGTGCTGGTGGTGGTTGGTGGAGTCCTGGCTTGCTATAGCTTGCTAGTAA
CAGTGGCCTTTATTATTTTCTGGGTGAGGAGTAAGAGGAGCAGGCTCCTGCACAGTG
ACTACATGAACATGACTCCCCGCCGCCCCGGGCCCACCCGCAAGCATTACCAGCCCT
ATGCCCCACCACGCGACTTCGCAGCCTATCGCTCCTGACACGGACGCCTATCCAGAA
GCCAGCCGGCTGGCAGCCCCCATCTGCTCAATATCACTGCTCTGGATAGGAAATGAC
CGCCATCTCCAGCCGGCCACCTCAGGCCCCTGTTGGGCCACCAATGCCAATTTTTCT
CGAGTGACTAGACCAAATATCAAGATCATTTTGAGACTCTGAAATGAAGTAAAAGA
GATTTCCTGTGACAGGCCAAGTCTTACAGTGCCATGGCCCACATTCCAACTTACCAT
GTACTTAGTGACTTGACTGAGAAGTTAGGGTAGAAAACAAAAAGGGAGTGGATTCT
GGGAGCCTCTTCCCTTTCTCACTCACCTGCACATCTCAGTCAAGCAAAGTGTGGTAT
CCACAGACATTTTAGTTGCAGAAGAAAGGCTAGGAAATCATTCCTTTTGGTTAAATG
GGTGTTTAATCTTTTGGTTAGTGGGTTAAACGGGGTAAGTTAGAGTAGGGGGAGGG
ATAGGAAGACATATTTAAAAACCATTAAAACACTGTCTCCCACTCATGAAATGAGCC
ACGTAGTTCCTATTTAATGCTGTTTTCCTTTAGTTTAGAAATACATAGACATTGTCTT
TTATGAATTCTGATCATATTTAGTCATTTTGACCAAATGAGGGATTTGGTCAAATGA
GGGATTCCCTCAAAGCAATATCAGGTAAACCAAGTTGCTTTCCTCACTCCCTGTCAT
GAGACTTCAGTGTTAATGTTCACAATATACTTTCGAAAGAATAAAATAGTTCTCCTA
CATGAAGAAAGAATATGTCAGGAAATAAGGTCACTTTATGTCAAAATTATTTGAGTA
CTATGGGACCTGGCGCAGTGGCTCATGCTTGTAATCCCAGCACTTTGGGAGGCCGAG
GTGGGCAGATCACTTGAGATCAGGACCAGCCTGGTCAAGATGGTGAAACTCCGTCT
GTACTAAAAATACAAAATTTAGCTTGGCCTGGTGGCAGGCACCTGTAATCCCAGCTG
CCCAAGAGGCTGAGGCATGAGAATCGCTTGAACCTGGCAGGCGGAGGTTGCAGTGA
GCCGAGATAGTGCCACAGCTCTCCAGCCTGGGCGACAGAGTGAGACTCCATCTCAA
ACAACAACAACAACAACAACAACAACAACAAACCACAAAATTATTTGAGTACTGTG
AAGGATTATTTGTCTAACAGTTCATTCCAATCAGACCAGGTAGGAGCTTTCCTGTTTC
ATATGTTTCAGGGTTGCACAGTTGGTCTCTTTAATGTCGGTGTGGAGATCCAAAGTG
GGTTGTGGAAAGAGCGTCCATAGGAGAAGTGAGAATACTGTGAAAAAGGGATGTTA
GCATTCATTAGAGTATGAGGATGAGTCCCAAGAAGGTTCTTTGGAAGGAGGACGAA
TAGAATGGAGTAATGAAATTCTTGCCATGTGCTGAGGAGATAGCCAGCATTAGGTG
ACAATCTTCCAGAAGTGGTCAGGCAGAAGGTGCCCTGGTGAGAGCTCCTTTACAGG
GACTTTATGTGGTTTAGGGCTCAGAGCTCCAAAACTCTGGGCTCAGCTGCTCCTGTA
CCTTGGAGGTCCATTCACATGGGAAAGTATTTTGGAATGTGTCTTTTGAAGAGAGCA
TCAGAGTTCTTAAGGGACTGGGTAAGGCCTGACCCTGAAATGACCATGGATATTTTT
CTACCTACAGTTTGAGTCAACTAGAATATGCCTGGGGACCTTGAAGAATGGCCCTTC
AGTGGCCCTCACCATTTGTTCATGCTTCAGTTAATTCAGGTGTTGAAGGAGCTTAGG
TTTTAGAGGCACGTAGACTTGGTTCAAGTCTCGTTAGTAGTTGAATAGCCTCAGGCA
AGTCACTGCCCACCTAAGATGATGGTTCTTCAACTATAAAATGGAGATAATGGTTAC
AAATGTCTCTTCCTATAGTATAATCTCCATAAGGGCATGGCCCAAGTCTGTCTTTGAC
TCTGCCTATCCCTGACATTTAGTAGCATGCCCGACATACAATGTTAGCTATTGGTATT
ATTGCCATATAGATAAATTATGTATAAAAATTAAACTGGGCAATAGCCTAAGAAGG
GGGGAATATTGTAACACAAATTTAAACCCACTACGCAGGGATGAGGTGCTATAATA
TGAGGACCTTTTAACTTCCATCATTTTCCTGTTTCTTGAAATAGTTTATCTTGTAATG
AAATATAAGGCACCTCCCACTTTTATGTATAGAAAGAGGTCTTTTAATTTTTTTTTAA
TGTGAGAAGGAAGGGAGGAGTAGGAATCTTGAGATTCCAGATCGAAAATACTGTAC
TTTGGTTGATTTTTAAGTGGGCTTCCATTCCATGGATTTAATCAGTCCCAAGAAGATC
AAACTCAGCAGTACTTGGGTGCTGAAGAACTGTTGGATTTACCCTGGCACGTGTGCC
ACTTGCCAGCTTCTTGGGCACACAGAGTTCTTCAATCCAAGTTATCAGATTGTATTTG
AAAATGACAGAGCTGGAGAGTTTTTTGAAATGGCAGTGGCAAATAAATAAATACTT
TTTTTTAAATGGAAAGACTTGATCTATGGTAATAAATGATTTTGTTTTCTGACTGGAA
AAATAGGCCTACTAAAGATGAATCACACTTGAGATGTTTCTTACTCACTCTGCACAG
AAACAAAGAAGAAATGTTATACAGGGAAGTCCGTTTTCACTATTAGTATGAACCAA
GAAATGGTTCAAAAACAGTGGTAGGAGCAATGCTTTCATAGTTTCAGATATGGTAGT
TATGAAGAAAACAATGTCATTTGCTGCTATTATTGTAAGAGTCTTATAATTAATGGT
ACTCCTATAATTTTTGATTGTGAGCTCACCTATTTGGGTTAAGCATGCCAATTTAAAG
AGACCAAGTGTATGTACATTATGTTCTACATATTCAGTGATAAAATTACTAAACTAC
TATATGTCTGCTTTAAATTTGTACTTTAATATTGTCTTTTGGTATTAAGAAAGATATG
CTTTCAGAATAGATATGCTTCGCTTTGGCAAGGAATTTGGATAGAACTTGCTATTTA
AAAGAGGTGTGGGGTAAATCCTTGTATAAATCTCCAGTTTAGCCTTTTTTGAAAAAG
CTAGACTTTCAAATACTAATTTCACTTCAAGCAGGGTACGTTTCTGGTTTGTTTGCTT
GACTTCAGTCACAATTTCTTATCAGACCAATGGCTGACCTCTTTGAGATGTCAGGCT
AGGCTTACCTATGTGTTCTGTGTCATGTGAATGCTGAGAAGTTTGACAGAGATCCAA
CTTCAGCCTTGACCCCATCAGTCCCTCGGGTTAACTAACTGAGCCACCGGTCCTCAT
GGCTATTTTAATGAGGGTATTGATGGTTAAATGCATGTCTGATCCCTTATCCCAGCC
ATTTGCACTGCCAGCTGGGAACTATACCAGACCTGGATACTGATCCCAAAGTGTTAA
ATTCAACTACATGCTGGAGATTAGAGATGGTGCCAATAAAGGACCCAGAACCAGGA
TCTTGATTGCTATAGACTTATTAATAATCCAGGTCAAAGAGAGTGACACACACTCTC
TCAAGACCTGGGGTGAGGGAGTCTGTGTTATCTGCAAGGCCATTTGAGGCTCAGAA
AGTCTCTCTTTCCTATAGATATATGCATACTTTCTGACATATAGGAATGTATCAGGAA
TACTCAACCATCACAGGCATGTTCCTACCTCAGGGCCTTTACATGTCCTGTTTACTCT
GTCTAGAATGTCCTTCTGTAGATGACCTGGCTTGCCTCGTCACCCTTCAGGTCCTTGC
TCAAGTGTCATCTTCTCCCCTAGTTAAACTACCCCACACCCTGTCTGCTTTCCTTGCT
TATTTTTCTCCATAGCATTTTACCATCTCTTACATTAGACATTTTTCTTATTTATTTGT
AGTTTATAAGCTTCATGAGGCAAGTAACTTTGCTTTGTTTCTTGCTGTATCTCCAGTG
CCCAGAGCAGTGCCTGGTATATAATAAATATTTATTGACTGAGTGAA.

As used herein, “T-cell function” refers to a T cell's (e.g., any of the exemplary T cells described herein) survival, stability, and/or ability to execute its intended function. For example, a CD4⁺ T cell can have an immunosuppressive function. A CD4⁺ T cell including a nucleic acid encoding a FOXP3 polypeptide can have a FOXP3-dependent expression profile that increases the immunosuppressive function of the T cell. For example, a cell transduced with a mutated FOXP3 polypeptide as described herein can have increased expression of genes that are transcriptional targets of a FOXP3 polypeptide that can result in increased Treg cell function. In some embodiments, a T cell is considered to have Treg function if the T cell exhibits or maintains the potential to exhibit an immune suppression function (e.g., an immunosuppressive phenotype).

As used herein, the term “cell surface marker” refers to polypeptides located at or on the surface of the cell such that at least a portion of the polypeptide is located at the exterior of the cell surface. As used herein, the term “cell surface expression profile” refers to one or more polypeptides located at or on the surface of the cell such that at least a portion of the polypeptide is located at the exterior of the cell surface that indicate a cell has a particular phenotype (e.g., an immunosuppressive phenotype).

As used herein, the term “control level” refers to the level (e.g., a level in a nucleus) of a corresponding wild type polypeptide in a corresponding mammalian cell.

As used herein, the term “activation” refers to induction of a signal on an immune cell (e.g., a B cell or T cell) that to results in initiation of the immune response (e.g., T cell activation).

As used herein, the term “stimulation” refers to stage of TCR or CAR signaling where a co-stimulatory signal can be used to achieve a robust and sustained TCR or CAR signaling response.

### Treg and Immunosuppressive Phenotypes

This document provides methods and materials for producing a T regulatory (Treg) cell. In a non-limiting example, a method for producing a Treg cell or an activated Treg cell includes contacting a T cell (e.g., a naïve T cell, a CD4+ T cell, a CD4+CD45RA+ T cell, a CD4+ CD62L+ T cell, or a central memory T cell) with an effective amount of (i) one or more CD3− stimulation agent(s) for a first period of time under conditions that allow for stimulation and activation of the T cell, and (ii) one or more agent(s) that decreases CD28 expression and/or activity; introducing into the T cell an effective amount of a nucleic acid sequence encoding a FOXP3 polypeptide, where the presence of the nucleic acid sequence in the T cell induces the T cell to develop or further develop one or more characteristics of a T regulatory cell phenotype compared to when the nucleic acid sequence is not present in the T cell; and contacting the T cell with an effective amount of interleukin-2 (IL-2) and/or TGF-β for a second period of time under conditions that allow for stabilization of a T regulatory cell phenotype as compared to when the T cell is not contacted with IL-2 and/or TGF-β for the second period of time.

In some embodiments, a Treg phenotype includes expression of CD4, CD25, CD127, and FOXP3. For example, a Treg phenotype can include expression of CD4⁺ CD25⁺ CD127^{lo} and FOXP3⁺. In some embodiments, a Treg phenotype includes a T cell having a cell surface expression profile of CD4⁺CD25⁺CD127^{lo/-}. Additional cell surface markers used to indicate a Treg phenotype include, without limitation, CD39 and CD73. For example, a T cell having a Treg phenotype includes a cell surface expression profile including CD4⁺ CD25⁺ CD127^{lo/-} CD39⁺CD73⁺. For example, a CD4⁺CD45RA⁺ T cell can develop into a CD4⁺CD45RA⁻ Treg cell using the methods described herein. In some embodiments, a Treg phenotype includes expression of latency-associated peptide (LAP), glycoprotein A repetitions predominant (GARP), and transforming growth factor beta 1 (TGF-β1). For example, a Treg cell can include a cell surface profile of CD4⁺CD25⁺LAP⁺, CD4⁺CD25⁺GARP⁺, or CD4⁺CD25⁺LAP⁺GARP⁺. In some embodiments, a suppressive phenotype in a Treg cell is confirmed by expression of one or more of CD25, CTLA-4, and GITR. In some embodiments, an immunosuppressive phenotype in a Treg cell is confirmed by a cytokine profile. For example, an immunosuppressive phenotype is confirmed in a Treg cell by low production of IL-2, IFN-gamma, and IL-17.

In some embodiments, flow cytometry is used to assess the T regulatory cell phenotype of a T cell produced according to any of the method described herein. For example, flow cytometry can be used to assess a T cell following contacting of the T cell with an effective amount of (i) one or more CD3-stimulation agent(s) for a first period of time under conditions that allow for stimulation and activation of the T cell, and (ii) one or more agent(s) that decreases CD28 expression and/or activity. In another example, flow cytometry can be used to assess the T regulatory phenotype following introduction of an effective amount of a nucleic acid sequence encoding a FOXP3 polypeptide. In other example, flow cytometry can be used to assess the T regulatory phenotype of a T cell following contacting the T cell with an effective amount of interleukin-2 (IL-2) and/or TGF-β for a second period of time. In yet another example, flow cytometry can be used to assess a T regulatory phenotype following introduction of a nucleic acid sequence encoding a truncated nerve growth factor receptor (tNGFR) polypeptide. In some embodiments, intracellular staining of a FOXP3 polypeptide is detected using flow cytometry. In some embodiments, flow cytometry is used to assess expression of a FOXP3 polypeptide and a cell surface expression profile (e.g., CD4 and CD25) of a T cell (e.g., any of the exemplary T cells provided herein).

In some embodiments, targeted next generation bisulfite sequencing (tNGS) is used to assess the phenotype of the T cell produced by the methods described herein (Floess et al. PLoS Biol. 5(2): e38 (2007)). Targeted next generation bisulfite sequencing enables evaluation of the methylation status of a customized panel of markers, including, but not limited to FOXP3. Methylation status of certain genes (e.g., hypomethylation) can be a marker of T regulatory cell. Difference between the naturally occurring Tregs (pTregs and tTregs) and iTregs is the epigenetic state of the genes including, without limitation, FOXP3, IL2RA, and CTLA4 in these cells. Regulatory DNA elements controlling the expression of these genes can be demethylated in tTregs and pTregs, while these same regulatory DNA elements are methylated in Tconv cells (Ohkura et al., Immunity, 37:785-99(2012)). In some cases, the CNS2 regulatory element of the FOXP3 gene, also referred to as the Treg-specific demethylated region (TSDR) can be methylated, thereby used to assess the phenotype of a T cell. Without wishing to be bound by theory, it is understood that methylated DNA regulatory sequences are associated with silenced genes, while demethylated DNA regulatory sequences are associated with actively transcribed genes (Cedar and Bergman, Nat. Rev. of Genet., 10:295-304 (2009)). In some cases, in iTregs produced by methods described in the art, the FOXP3 CNS2 regulatory element is methylated. In some cases, a demethylated FOXP3 TSDR (e.g., the CNS2 regulatory element) is associated with stability of the Treg phenotype (see, e.g., Feng et al., Cell, 158: 749 (2014)).

Methods of Introducing Nucleic Acids and Polypeptides into T Cells

In some embodiments, a cell (e.g., a T cell) that is transduced with the nucleic acid sequences described herein is isolated from a mammal (e.g., a human) using any appropriate method (e.g., magnetic activated sorting or flow cytometry-mediated sorting). In some cases, a nucleic acid sequence encoding a FOXP3 polypeptide can be transformed into a T cell using a lentivirus and then cultured according to the methods described herein. In some cases, a nucleic acid sequence encoding an siRNA or an shRNA can be transformed into a T cell using a lentivirus and then cultured according to the methods described herein. In some cases, nucleic acid sequences encoding a FOXP3 polypeptide can be transformed into a T cell along with nucleic acid sequences encoding siRNA or shRNA and/or a tNGFR polypeptide. For example, a Treg cell can be made by transducing nucleic acid sequences encoding a FOXP3 polypeptide and a siRNA or shRNA into a T cell using a lentivirus and then culturing the T cell according to the methods described herein. In another example, a Treg cell can be made by transducing nucleic acid sequences encoding a FOXP3 polypeptide and a tNGFR polypeptide into a T cell (e.g., a T cell) using a lentivirus and then culturing the T cell according to the methods described herein. In yet another example, a Treg cell can be made by transducing nucleic acid sequences encoding a FOXP3 polypeptide, a siRNA or shRNA, and a tNGFR polypeptide into a T cell using a lentivirus and then culturing the T cell according to the methods described herein. In all cases described herein, the nucleic acid sequences are operably linked to a promoter or are operably linked to other nucleic acid sequences using a self-cleaving 2A polypeptide or IRES sequence.

Methods of introducing nucleic acids and expression vectors into a cell (e.g., an eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection. As used herein, “transformed” and “transduced” are used interchangeably.

In some embodiments, a resting Treg cell (e.g., a CD4⁺CD45RA⁺Foxp3^{lo} T cell) can be converted to an activated Treg cell (e.g., a CD4⁺ CD25⁺ Foxp3⁺ T cell) using the methods described herein. In some embodiments, a naive Treg cell (e.g., a CD4⁺ CD45RA⁻Foxp3⁻ T cell) can be converted to an activated Treg cell (e.g., a CD4⁺ CD45RA⁻Foxp3⁺ T cell) using any of the methods described herein. In some embodiments, a non Treg cell (e.g., a CD4⁺ CD45 RA⁺ Foxp3^{lo} T cell) can be converted to an activated Treg cell (e.g., a CD4⁺ CD45RA⁺ Foxp3⁺ T cell using any of the methods described herein.

Also provided herein are methods of transducing a T cell where the method includes: (a) contacting a T cell with an effective amount of (i) one or more CD3-stimulation agent(s) in the absence of a CD28 stimulating agent for a first period of time under conditions that allow for stimulation and activation of the T cell, and (b) introducing into the T cell an effective amount of a nucleic acid sequence encoding one or more polypeptides operatively linked to a promoter active in T cells, thereby transducing the T cell. In some embodiments, the method includes contacting the T cell with an effective amount of one or more agent(s) that decreases CD28 expression and/or activity. Some embodiments of these methods result in at least a 1% increase, at least a 2% increase, at least a 3% increase, at least a 4% increase, at least a 5% increase, at least a 6% increase, at least a 7% increase, at least a 8% increase, at least a 9% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 120% increase, at least a 140% increase, at least a 160% increase, at least a 180% increase, or at least a 200% increase, in the number of transduced T cells (e.g., as compared to the number of transduced cells when the T cells are not activated or are activated with methods using CD3/CD28 prior to transduction).

Also provided herein are methods of transducing a T cell where the method includes: (a) contacting a T cell with an effective amount of (i) one or more CD3-stimulation agent(s), and (ii) one or more CD28-stimulation agent(s) for a first period of time under conditions that allow for stimulation and activation of the T cell; (b) contacting the T cell with an effective amount of one or more agent(s) that decreases CD28 expression and/or activity; and (c) introducing into the T cell an effective amount of a nucleic acid sequence encoding one or more polypeptides operatively linked to a promoter active in T cells, thereby transducing the T cell. Some embodiments of these methods result in at least a 1% increase, at least a 2% increase, at least a 3% increase, at least a 4% increase, at least a 5% increase, at least a 6% increase, at least a 7% increase, at least a 8% increase, at least a 9% increase, at least a 10% increase, at least a 15% increase, at least a 20% increase, at least a 25% increase, at least a 30% increase, at least a 35% increase, at least a 40% increase, at least a 45% increase, at least a 50% increase, at least a 55% increase, at least a 60% increase, at least a 65% increase, at least a 70% increase, at least a 75% increase, at least a 80% increase, at least a 85% increase, at least a 90% increase, at least a 95% increase, at least a 100% increase, at least a 120% increase, at least a 140% increase, at least a 160% increase, at least a 180% increase, or at least a 200% increase, in the number of transduced T cells (e.g., as compared to the number of transduced cells when the T cells are not activated or are activated with methods using CD3/CD28 prior to transduction).

### Nucleic Acids/Vectors

Also provided herein are nucleic acids sequences that encode for any of the polypeptides described herein. Also provided herein are nucleic acid sequences that encode for one or more of a short interfering RNA (siRNA) or a short hairpin RNA (shRNA). A nucleic acid sequence can include: a nucleic acid sequence encoding a FOXP3 polypeptide; a nucleic acid sequence encoding a FOXP3 polypeptide and a nucleic acid sequence encoding one or more siRNA or shRNA; a nucleic acid sequence encoding a FOXP3 polypeptide and a nucleic acid sequence encoding a tNGFR polypeptide; or a nucleic acid sequence encoding a FOXP3 polypeptide, a nucleic acid sequence encoding a tNFGR polypeptide, and a nucleic acid sequence encoding one or more siRNA or shRNA. Also provided herein are vectors that include any of the nucleic acids encoding any of the polypeptides described herein and any of the siRNA or shRNA described herein, or any combinations thereof. For example, a vector can include: a nucleic acid sequence encoding a FOXP3 polypeptide and a nucleic acid sequence encoding one or more siRNA or shRNA; a nucleic acid sequence encoding a FOXP3 polypeptide and a nucleic acid sequence encoding a tNGFR polypeptide; or a nucleic acid sequence encoding a FOXP3 polypeptide, a nucleic acid sequence encoding a tNFGR polypeptide, and a nucleic acid sequence encoding one or more siRNA or shRNA, where one or more of the nucleic acid sequences is operably linked to a promoter.

In some embodiments, the methods provided herein include introducing into a T cell a nucleic acid sequence encoding one or more polypeptides. In some embodiments, the one or more polypeptides are one or more exogenous polypeptides (e.g., one or more non-naturally occurring polypeptides). In some embodiments, the one or more polypeptides can include one or more cytokines, cytokine receptors, differentiation factors, growth factors, growth factor receptors, peptide hormones, metabolic enzymes, receptors, T cell receptors, chimeric antigen receptors (CARs), transcriptional activators, transcriptional repressors, translation activators, translational repressors, immune-receptors, apoptosis inhibitors, apoptosis inducers, immune-activators, and immune-inhibitors, or any combination thereof. In some embodiments, the polypeptide can be a CAR. In some embodiments, a chimeric antigen receptor (CAR) as described herein includes an extracellular antigen-binding domain, a transmembrane domain, and an intracellular domain (e.g., that includes one or more intracellular co-stimulatory domains, e.g., a CD3 zeta intracellular co-stimulatory domain). In some embodiments, the extracellular domain includes an antigen-binding domain that is capable of binding to a cluster of differentiation 19 (CD19) molecule. An exemplary CD19 polypeptide includes without limitation amino acid sequence corresponding to NCBI reference sequences: NP_001171569.1, P_001372661.1, and NP_001761.3. Non-limiting examples of CARs that include an extracellular antigen-binding domain that binds specifically to CD19 are described in US 2021/0060080, US 2021/0060079, US 2021/0002366, US 2021/0000869, US 2020/0392248, US 2020/0360431, US 2020/0140544, US 2020/0030424, US 2019/0388471, US 2019/0292238, US 2019/0209616, US 2019/0135940, US 2018/0153977, US 2018/0044417, US 2016/0362472, US 2016/0145337, US 2015/0283178, and US 2014/0271635 (each incorporated herein by reference). In some embodiments, the nucleic acid sequence encoding one or more polypeptides (e.g., one or more exogenous polypeptides) is present in a nucleic acid construct or vector (e.g., any of the exemplary nucleic acid vectors, e.g., viral vectors, described herein).

Any of the vectors described herein can be an expression vector. For example, an expression vector can include a promoter sequence operably linked to the sequence encoding any of the polypeptides and/or siRNA or shRNA as described herein. Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. In some cases, a vector can include sufficient cis-acting elements that supplement expression where the remaining elements needed for expression can be supplied by the host mammalian cell or in an in vitro expression system. Skilled practitioners will be capable of selecting suitable vectors and mammalian cells for making any of the T cells as described herein. Any appropriate promoter (e.g., EF1 alpha) can be operably linked to any of the nucleic acid sequences described herein. Non-limiting examples of promoters to be used in any of the vectors or constructs described herein include EF1a, SFFV, PGK, CMV, CAG, UbC, MSCV, MND, EFla hybrid, and/or CAG hybrid. In some embodiments, the nucleic acid sequence encoding one or more polypeptides is operatively linked to a promoter active in T cells (e.g., any of the exemplary promoters described herein). As used herein, the term “operably linked” is well known in the art and refers to genetic components that are combined such that they carry out their normal functions. For example, a gene is operably linked to a promoter when its transcription is under the control of the promoter. In another example, a nucleic acid sequence can be operably linked to other nucleic acid sequence by a self-cleaving 2A polypeptide or an internal ribosome entry site (IRES). In such cases, the self-cleaving 2A polypeptide allows the second nucleic acid to be under the control of the promoter operably linked to the first nucleic acid sequence. The nucleic acid sequences described herein can be operably linked to a promoter. In some cases, the nucleic acid sequences described herein can be operably linked to any other nucleic acid sequence described herein using a self-cleaving 2A polypeptide or IRES. In some cases, the nucleic acid sequences are all included on one vector and operably linked either to a promoter upstream of the nucleic acid sequences or operably linked to the other nucleic acid sequences through a self-cleaving 2A polypeptide or an IRES.

### Compositions

In some embodiments, the compositions include any of the cells (e.g., any of the cells described herein including any of the cells produced using any of the methods described herein). In some embodiments, the compositions include any of the vectors (e.g., any of the vectors described herein including any nucleic acid sequences described herein). In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

### Kits

Also provided herein are kits that include any of T cells or vectors described herein, any of the compositions described herein, or any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

### Cells

Also provided herein are cells (e.g., any of the exemplary cells described herein or known in the art) comprising any of the nucleic acids described herein that encode any of the polypeptides (e.g., FOXP3 polypeptides and tNGFR polypeptide) described herein and any of the siRNA or shRNA described herein. Also provided herein are cells (e.g., any of the exemplary cells described herein or known in the art) that include any of the vectors described herein that encode any of the polypeptides (e.g., FOXP3 polypeptides and tNGFR polypeptide) described herein and any of the siRNA or shRNA described herein.

In some embodiments, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

In some embodiments, the T cell is an autologous T cell obtained from the subject (e.g., the mammal to be treated). In some embodiments, the resting Treg cell (e.g., a CD4⁺ CD45RA⁺Foxp3^{lo} T cell) is an autologous resting Treg cell obtained from the subject. In some embodiments, the naive Treg cell (e.g., a CD4⁺ CD45RA⁻ Foxp3⁻ T cell) is an autologous naïve Treg cell obtained from the subject. In some embodiments, the non Treg cell (e.g., a CD4⁺ CD45RA⁺Foxp3^{lo} T cell) is an autologous non Treg T cell obtained from the subject.

In some embodiments, the T cells are obtained from an allogeneic source of T cells. In some embodiments, the T cells are obtained from a pluripotent stem cell via a directed differentiation protocol. In some embodiments, the T cells are genetically genetically-engineered prior to the introduction of a first nucleic sequence and a second nucleic acid sequence.

### Methods of Treatment

Also provided herein are methods of treating a mammal (e.g., a human) having an autoimmune disease that includes administering to the mammal (e.g., human) a therapeutically effective amount of a T regulatory cell produced according to any of the methods described herein. Additional methods provided include a method of treating a mammal (e.g., a human) having an autoimmune disease that includes administering to the mammal (e.g., human) a therapeutically effective amount of any of the compositions (e.g., pharmaceutical compositions) described herein.

In some embodiments, these methods can result in a reduction in the number, severity, or frequency of one or more symptoms of the autoimmune diseases in the mammal (e.g., as compared to the number, severity, or frequency of the one or more symptoms of the autoimmune disease in the mammal prior to treatment). For example, a mammal having an autoimmune disease having been administered a T cell as described here can experience a reduction in inflammation or autoantibody production.

A pharmaceutical composition containing the T cells and a pharmaceutically acceptable carrier can be administered to a mammal (e.g., a human) having an autoimmune disease. For example, a pharmaceutical composition (e.g., a T cell along with a pharmaceutically acceptable carrier) to be administered to a mammal having an autoimmune disease can be formulated in an injectable form (e.g., emulsion, solution and/or suspension).

Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Effective dosage of T cells can vary depending on the severity of the autoimmune disease, the route of administration, the age and general health condition of the subject, excipient usage, the possibility of co-usage with other therapeutic treatments, and the judgment of the treating physician. An effective amount of a T cell can be any amount that reduces inflammation and autoantibody production within a mammal having an autoimmune disease without producing significant toxicity to the mammal. For example, an effective amount of T cells administered to a mammal having an autoimmune disease can be from about 1×10⁶ cells to about 1×10¹⁰ (e.g., from about 1×10⁶ to about 1×10⁹, from about 1×10⁶ to about 1×10⁸, from about 1×10⁶ to about 1×10⁷, from about 1×10⁷ to about 1×10¹⁰, from about 1×10⁷ to about 1×10⁹, from about 1×10⁷ to about 1×10⁸, from about 1×10⁸ to about 1×10¹⁰, from about 1×10⁸ to about 1×10⁹, or form about 1×10⁹ to about 1×10¹⁰) cells. In some cases, the T cells can be a purified population of immune cells generated as described herein. In some cases, the purity of the population of T cells can be assessed using any appropriate method, including, without limitation, flow cytometry. In some cases, the population of T cells to be administered can include a range of purities from about 70% to about 100%, from about 70% to about 90%, from about 70% to about 80%, from about 80% to about 90%, from about 90% to about 100%, from about 80% to about 100%, from about 80% to about 90%, or from about 90% to 100%. In some cases, the dosage (e.g., number of T cells to be administered) can adjusted based on the level of purity of the T cells.

The frequency of administration of a T cell (e.g., any of the T cells described herein) can be any frequency that reduces inflammation or autoantibody production within a mammal having an autoimmune disease without producing toxicity to the mammal. In some cases, the actual frequency of administration of a T cell can vary depending on various factors including, without limitation, the effective amount, duration of treatment, use of multiple treatment agents, route of administration, and severity of the condition may require an increase or decrease in frequency of administration.

An effective duration for administering a composition containing a T cell can be any duration that reduces inflammation or autoantibody production within a mammal having an autoimmune disease without producing toxicity to the mammal. In some cases, the effective duration can vary from several days to several months to several years. In general, the effective treatment duration for administering a composition containing a T cell to treat an autoimmune disease can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some cases, the effective treatment duration for administering a composition containing a T cell can be for the remainder of the life of the mammal.

In some cases, a course of treatment and/or the severity of one or more symptoms related to autoimmune disease can be monitored. Any appropriate method can be used to determine whether the autoimmune disease is being treated. For example, immunological techniques (e.g., ELISA) can be performed to determine if the level of autoantibodies present within a mammal being treated as described herein is reduced following the administration of the T cells. Remission and relapse of the disease can be monitored by testing for one or more markers of autoimmune disease.

Any appropriate autoimmune disease can be treated with a T cell as described herein. In some cases, an autoimmune disease caused by the accumulation of autoantibodies can be treated with a T cell as described herein. Examples of autoimmune diseases include, without limitation, Inflammatory Bowel Disease, Lupus, Rheumatoid Arthritis, Multiple Sclerosis, Insulin Dependent Diabetes Mellitis, Myasthenia Gravis, Grave's disease, Autoimmune Hemolytic Anemia, Autoimmune Thrombocytopenia Purpura, Goodpasture's Syndrome, Pemphigus Vulgaris, Acute Rheumatic Fever, Post-Streptococcal Glomerulonephritis, Crohn's Disease, Celiac Disease, and Polyarteritis Nodosa.

The invention will be further described in the following examples, which do not limit the scope of the invention described in the claims.

### EXAMPLES

### Example 1. A Method for Producing T Regulatory Cells

Frozen naïve human CD4⁺ (CD3⁺CD4⁺CD45RA⁺CD45RO⁻) were thawed and rested overnight in Immunocult complete (ICC) media (serum-free Immunocult XF T cell expansion media supplemented with Glutamax, sodium pyruvate, MEM-NEAA, HEPES, Penicillin-Streptomycin, and 2-mercaptoethanol). T cells were activated by anti-CD3 antibody (CD3 activation) or anti-CD3 and anti-CD28 antibodies (CD3/CD28 activation). CD3 activation was accomplished with Biolegend ultra leaf purified anti-human CD3 antibody immobilized on tissue culture plates. Tissue culture plates were coated with immobilized anti-CD3 antibody by incubating with 200 uL of a 10 ug/mL solution of anti-human CD3 antibody overnight at 4° C. Following incubation, coated anti-CD3 wells were decanted and washed three times with PBS. CD3/CD28 activation was performed by adding 25 uL of Immunocult Human CD3/CD28 T cell Activator (STEMCELL Technologies) per mL of cell suspension. Defrosted T cells were counted, checked for viability, and then re-suspended in ICC media at a concentration of 1×10⁶ cells/mL. Cells were activated for 24 hours at 1×10⁶ cells/mL in a volume of 450 uL using either CD3 activation or CD3/CD28 activation in the presence of media alone, 10 ng/mL IL-2 (STEMCELL Technologies), 1 ng/mL TGFβ (R&D Systems), or IL-2 and TGFβ.

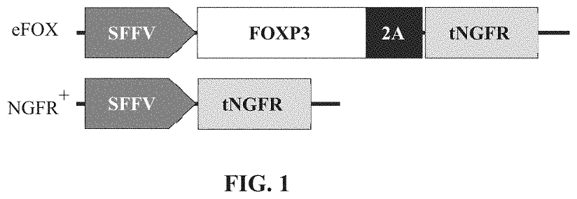

Cells were transduced with lentiviral vectors encoding either human FOXP3 and truncated NGFR (tNGFR) or tNGFR alone under the control of an SSFV promoter at a multiplicity of infection (MOI) of 50 (FIG. 1). Briefly, lentiviruses were produced by transfecting suspension HEK293 cells with Lenti-Packaging Plasmid Mix (Cellecta) and the lentiviral vector (e.g., vector expression FOXP3 or FOXP3+tNFGR) plasmid using ExpiFectamine (Invitrogen) transfection reagent. Cells were clarified and filtered on day 4, followed by addition of 4× Peg-It solution (System Biosciences) to the lentivirus supernatant and incubation at 4° C. The following day the supernatant mix was spun at high speed for 1 hour to pellet the virus. The viral pellet was then re-suspended followed by resuspension of virus pellet in T cell media. Viral titers were obtained using Go-Stix kit from Takara Bio. Optionally, re-suspended viral pellets were flash frozen for future use.

T Cells were cultured at 37° C. in 5% CO2 for 12 days. Fresh ICC media, supplemented with 10 ng/mL IL-2 was added to cells every 2-3 days starting at day 2 post transduction in order to maintain cell density at ˜0.5×10⁶ cells/mL. Cells were maintained in culture for 12 days, with re-stimulation with Immunocult T Cell Activator on day 8 (5 uL per mL cells). FOXP3 expression and other Treg phenotypic markers were assessed by flow cytometry at days 4 and 12 post transduction using the antibodies provided in Table 2. Transduced cells were purified using Miltenyi MACS LS columns with MACSelect LNGFR Microbeads for NGFR-positive selection on Day 4 or 5 post transduction.

Cell pellets from days 5 and 12 post transduction were analyzed by targeted next generation bisulfite sequencing (tNGS) to evaluate the methylation status of transduced cells using a customized human FOXP3 Treg panel by EpigenDx, Inc.

TABLE 2
Antibodies used for flow cytometry

| Antibobies | Vendor | Cat # | Clone # |
|---|---|---|---|
| NGFR BB515 | BD | 564580 | C40-1457 |
| FoxP3 PE-CF594 | BD | 562421 | 259D/C7 |
| CTLA4 PE-Cy7 | Biolegend | 369614 | BNI3 |
| CD25 BV421 | BD | 564033 | 2A3 |
| Live/Dead eFl 506 | eBioscience | 65-0866-14 | 2010926 |
| CD4 BUV496 | BD | 612936 | SK3 |

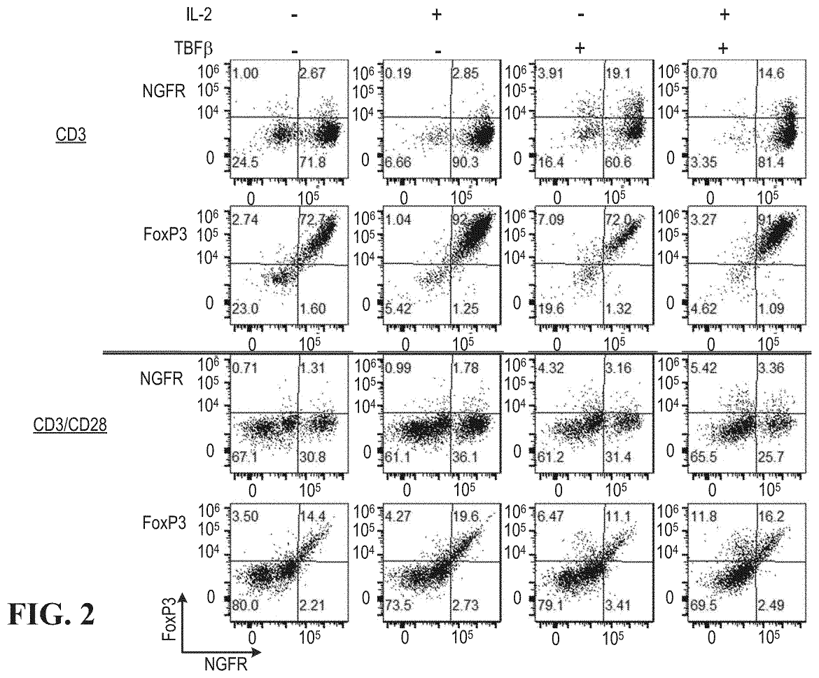

Analysis of cells by flow cytometry on day 4 post-transduction indicated that transduction efficiency was significantly higher in cells activated by CD3 activator than in cells activated by CD3/CD28 activator (FIG. 2). This surprising result was evident in both the levels of FOXP3 and tNGFR expression at Day 4. CD3 activated T cells were 74-96% NGFR⁺ and 75-93% FOXP3⁺, while CD3/CD28 activated T cells were 29-38% NGFR⁺ and 11-20% FoxP3⁺ for CD3/CD28 activated T cells.

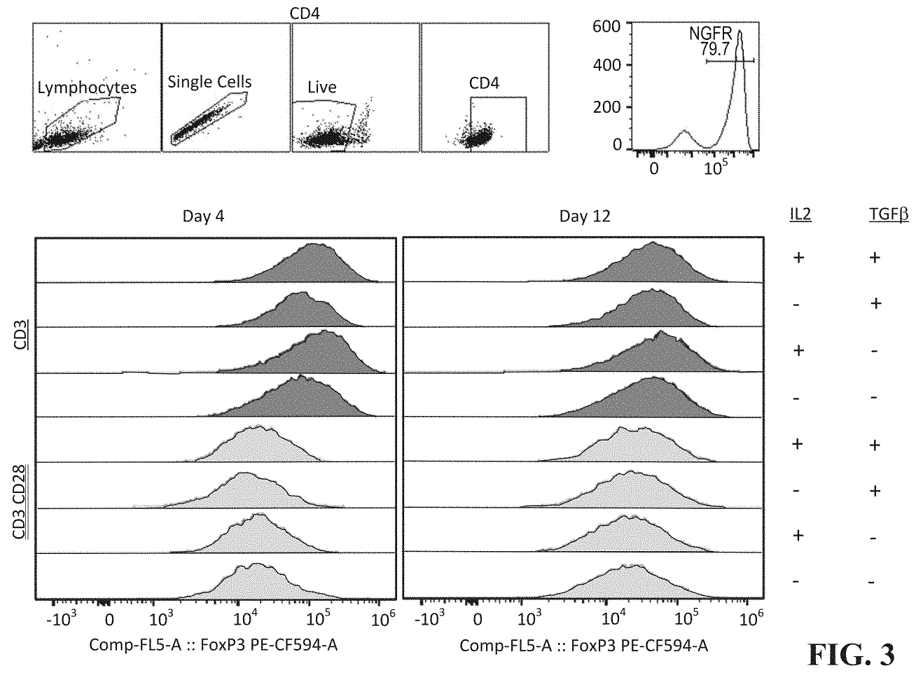

In addition to the higher percentage of FOXP3⁺ cells in the CD3 activated condition, these cells exhibited higher FOXP3 mean fluorescence intensity (MFI) compared to the T cells activated with CD3/CD28. MFIs of CD3 activated cells were higher on day 4 and remained higher on day 12 (FIG. 3).

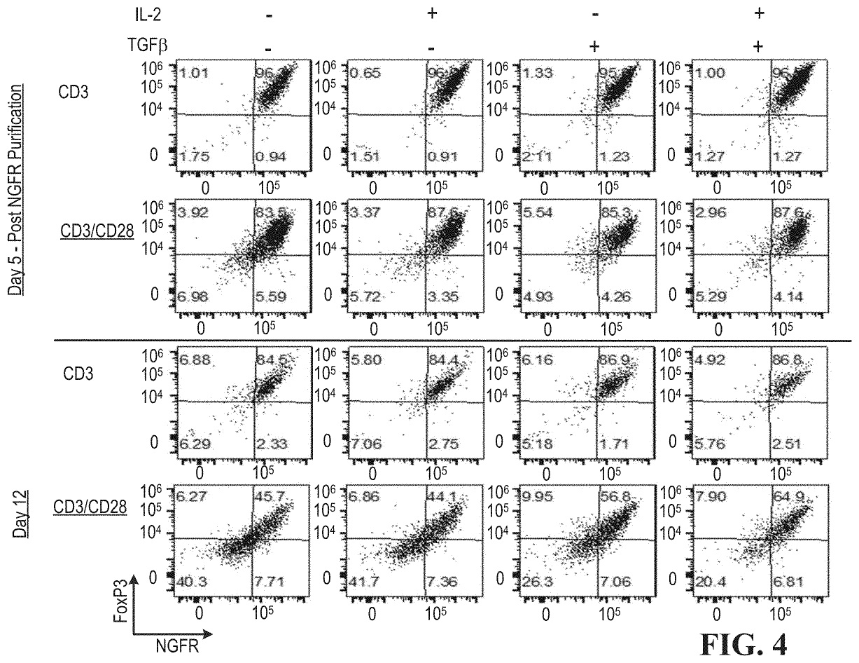

These results show that CD3 activation prior to lentivirus transduction was highly favorable for stable lentiviral transduction of naïve T cells, resulting in high FOXP3 expression and maintenance of high FOXP3 expression at day 12 post transduction. Transduction of naïve T cells with FOXP3-Lentivirus post CD3/CD28 activation results in a mixture of transduced and untransduced cells T cells and the percentage of FOXP3⁺ cells rapidly declines over time. This decline is due to outgrowth of untransduced cells, possible loss of transduced FOXP3 expression, or both. CD3 activated cultures transduced with FOXP3-tNGFR-Lentivirus maintained a significantly higher percentage of FOXP3⁺ cells than CD3/CD28 activated culture. The percentage of FOXP3⁺ cells decreased by ˜10% in CD3 activated T cells between day 5 and day 12, while in CD3/CD28 activated T cells the percentage of FOXP3⁺ cells decreased by ˜40% (FIG. 4).

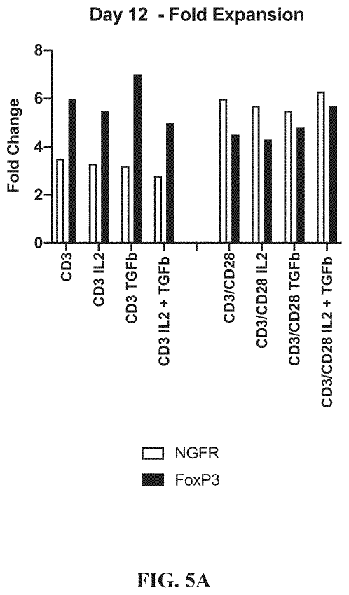

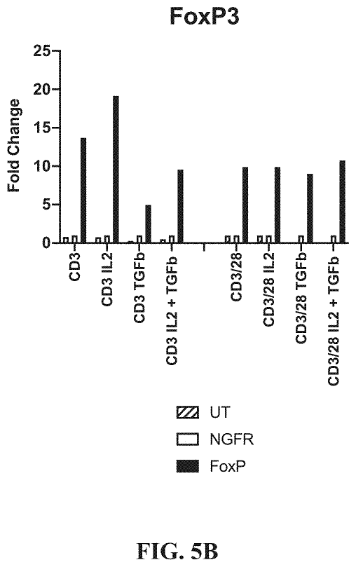

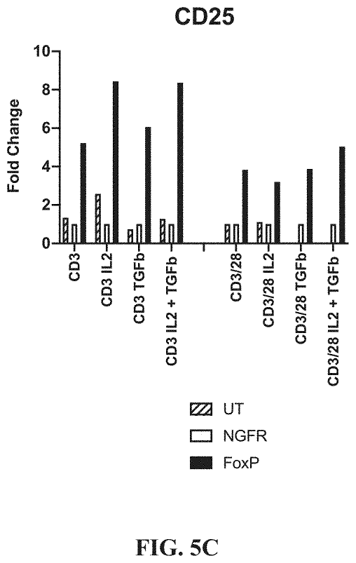

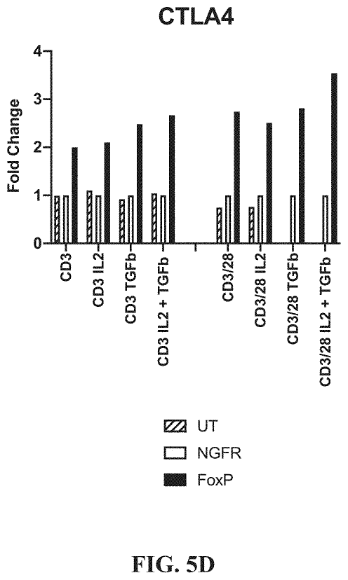

CD3 activation may also confer a growth advantage to FOXP3⁺ cells over untransduced cells, thereby limiting outgrowth of untransduced cells typically observed in Treg cultures with naïve CD4+ cells. Additionally, at day 8 post re-stimulation, the expansion of FOXP3⁺ cells was similar in CD3 activated T cells and CD3/CD28 activated T cells (FIG. 5A), while the expansion of FOXP3⁻ cells was lower. CD3 activated T cells also maintained similar profiles to CD3/CD28 activated T cells for distinctive markers of Treg phenotype (FIGS. 5B-5D).

Increased transduction efficiency (as measured on day 4 post-transduction) and increased FOXP3 expression was observed in CD3 activated cells irrespective of whether they were also cultured in media alone, IL-2, TGFβ, or IL-2+TGFβ during the activation period (FIG. 2). These results were unexpected based on the findings of Mikami et al. (PNAS 117(22): 12258-12268; 2020), who found that increased generation of iTregs from Tconv cells in mouse cells required both CD3 activation, IL-2, and TGFβ.

### Example 2: Additional Strategies for Producing T Regulatory Cells

Other strategies may be employed to similarly produce engineered Tregs. All commercially available reagents for activation of T cells include both anti-CD3 and anti-CD28 antibodies. Addition of CD3/CD28 activation reagents to T cells in the presence of a blocking CD28 antibody, Fab or scFv antibody fragments, or other CD28 blocking agents may be used to limit the T cell activation to CD3 activation alone. A CD28 blocking agent can be included during the first 48 hour of T cell stimulation and activation to enable CD3 activation, and thereafter removed during further culture to restore CD28 signaling for the remainder of the culture process. These cell culture conditions would mimic a CD3-only activation condition. An alternative strategy is to activate naïve T cells in the presence of a CD3/CD28 activation reagent and to transduce the cells with a lentiviral vector encoding both FOXP3 and a shRNA directed to CD28 to silence CD28 signaling following transduction.

### Example 3: A Method for Producing Chimeric Antigen Receptor T Cells

Frozen bulk CD3⁺(CD3⁺CD4⁺ and CD3⁺CD8⁺) and naïve human CD4⁺ (CD3⁺CD4⁺CD45RA⁺CD45RO⁻) were thawed and rested overnight in Immunocult complete (ICC) media (serum-free Immunocult XF T cell expansion media supplemented with Glutamax, sodium pyruvate, MEM-NEAA, HEPES, Penicillin-Streptomycin, and 2-mercaptoethanol). T cells were activated using an anti-CD3 antibody (CD3 activation), anti-CD3 and anti-CD28 antibodies (CD3/CD28 activation, ImmunoCult), or humanized CD3 and CD28 agonists (CD3/CD28 activation, TransAct). CD3 activation was accomplished with BioLegend® Ultra-LEAF™purified anti-human CD3 antibody immobilized on tissue culture plates. Tissue culture plates were coated with immobilized anti-CD3 antibody by incubating with 200 μL of a 10 μg/mL solution of anti-human CD3 antibody overnight at 4° C. Following incubation, coated anti-CD3 wells were decanted and washed three times with PBS. CD3/CD28 activation (ImmunoCult) was performed by adding 25 μL of Immunocult Human CD3/CD28 T cell Activator (STEMCELL Technologies) per mL of cell suspension. CD3/CD28 activation (TransAct) was performed by adding 10 μL of T Cell TransAct™, human (Miltenyi Biotec) per mL of cell suspension. Frozen cells were thawed and rested overnight in ICC media. The following day, cells were counted, checked for viability, and then re-suspended in ICC media at a concentration of 1×10⁶ cells/mL. Cells were activated for 24 hours at 1×10⁶ cells/mL in a volume of 200 μL using either CD3 activation, CD3/CD28 activation (ImmunoCult), or CD3/CD28 activation (TransAct).

T cells were transduced with lentiviral vectors encoding a second generation anti-CD19 chimeric antigen receptor (CAR) under the control of an SFFV promoter at a multiplicity of infection (MOI) of 5. Briefly, lentiviruses were produced by transfecting suspension HEK293 cells with Lenti-Packaging Plasmid Mix (Cellecta) and the lentiviral vector (e.g., vector encoding the anti-CD19 CAR) using ExpiFectamine (Invitrogen) transfection reagent. Cells were clarified and filtered on day 4, followed by addition of 4×PEG-it virus precipitation solution (System Biosciences) to the lentivirus supernatant and incubation at 4° C. The following day the supernatant mix was spun at high speed for 1 hour to pellet the virus. The viral pellet was then re-suspended in T cell media. Optionally, re-suspended viral pellets were flash frozen prior to being used for infecting T cells. Viral titers were obtained by infecting K562 cells with a 3-fold dilution series prepared from a thawed aliquot of the virus batch (previously flash frozen). Each dilution in culture media was prepared such that the same volume was added to each well containing 1×10⁵ K562 cells. Culture media was added to each well the following day, and the entirety of each well was harvested for flow cytometry to determine transgene expression 3 days post-infection.

Following lentiviral transduction, T cells were cultured at 37° C. in 5% C02 for 4 days. Fresh ICC media, supplemented with 10 ng/mL IL-2, was added to cells at day 2 post transduction in order to maintain cell density at ˜0.5×10⁶ cells/mL. Anti-CD19 CAR expression was assessed by flow cytometry at day 4 post transduction using the reagents provided in Table 3.

TABLE 3
Reagents used for flow cytometry

| Reagent | Vendor | Cat # | Clone # |
|---|---|---|---|
| Recombinant Protein L, PE | Sino | 11044-H07E-P | N/A |
| | Biological | | |
| Mouse Anti-Human CD3, | BD | 564712 | HIT3α |
| BV605 | Horizon | | |
| Mouse Anti-Human CD4, | BD | 560649 | RPA-T4 |
| PE-Cy7 | Pharmingen | | |
| Mouse Anti-Human CD8, | BD | 560662 | RPA-T8 |
| PerCP-Cy5.5 | Pharmingen | | |
| Fixable Viability Dye | Invitrogen | 65-0865-14 | N/A |
| eFluor ™ 780 | | | |

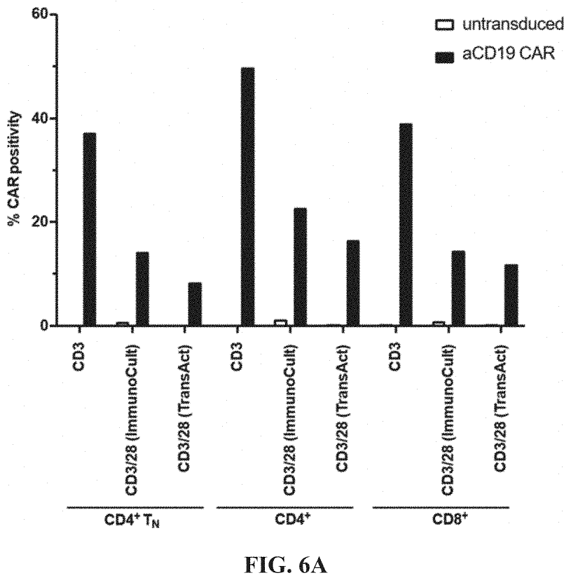

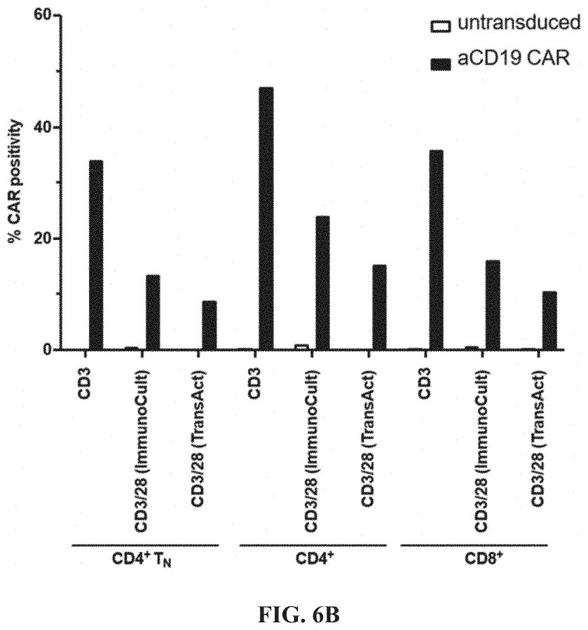

Analysis of cells by flow cytometry on day 4 post-transduction indicated that transduction efficiency was significantly higher in T cells activated by CD3 activator than in T cells activated by either of the CD3/CD28 activator methods (see FIG. 6A (donor 1) and FIG. 6B (donor 2)). The significantly higher transduction efficiency was observed in the levels of anti-CD19 CAR expression at Day 4 for both naïve human CD4⁺(CD4⁺ T_{N}) and bulk CD3⁺ T cells (split into CD4⁺ and CD8⁺ main subsets). At Day 4, T cells previously activated with CD3 were 37-50% CAR⁺ (Donor 1; see FIG. 6A) and 34-47% CAR⁺ (Donor 2; see FIG. 6B), while T cells previously activated with CD3/CD28 (ImmunoCult) were 14-23% CAR⁺ (Donor 1; see FIG. 6A) and 14-24% CAR⁺ (Donor 2; see FIG. 6B) and T cells previously activated with CD3/CD28 (TransAct) were 8-17% CAR⁺ (Donor 1; see FIG. 6A) and 9-15% CAR⁺ (Donor 2; see FIG. 6B). These results show that CD3 activation prior to lentivirus transduction resulted in increased lentiviral transduction of naïve CD4⁺ and bulk CD3⁺ T cells, resulting in high CAR expression as compared to T cells previously activated with either CD3/CD28 (ImmunoCult) or CD3/CD28 (TransAct).

### EMBODIMENTS

Embodiment 1 is a method of producing T regulatory cells, comprising: (a) contacting a T cell with an effective amount of (i) one or more CD3-stimulation agent(s) in the absence of a CD28 stimulating agent for a first period of time under conditions that allow for stimulation and activation of the T cell, and (b) introducing into the T cell an effective amount of a nucleic acid sequence encoding a forkhead box protein 3 (FOXP3) polypeptide, wherein the presence of the nucleic acid sequence in the T cell induces the T cell to develop or further develop one or more characteristics of a T regulatory cell phenotype compared to when the nucleic acid sequence is not present in the T cell.

Embodiment 2 is the method of embodiment 1, further comprising contacting the T cell with an effective amount of one or more agent(s) that decreases CD28 expression and/or activity.

Embodiment 3 is a method of producing T regulatory cells, comprising: (a) contacting a T cell with an effective amount of (i) one or more CD3-stimulation agent(s), and (ii) one or more CD28-stimulation agent(s) for a first period of time under conditions that allow for stimulation and activation of the T cell; (b) contacting the T cell with an effective amount of one or more agent(s) that decreases CD28 expression and/or activity; and (c) introducing into the T cell an effective amount of a nucleic acid sequence encoding a forkhead box protein 3 (FOXP3) polypeptide, wherein the presence of the nucleic acid sequence in the T cell induces the T cell to develop or further develop one or more characteristics of a T regulatory cell phenotype compared to when the nucleic acid sequence is not present in the T cell.

Embodiment 4 is the method of any one of embodiment 1-3, further comprising contacting the T cell with an effective amount of interleukin-2 (IL-2) and/or TGF-β for a second period of time under conditions that allow for stabilization of a T regulatory phenotype as compared to when the T cell is not contacted with IL-2 and/or TGF-β for the second period of time.

Embodiment 5 is the method of any one of embodiments 1-3, wherein the method does not comprise contacting the T cell with IL-2.

Embodiment 6 is the method of any one of embodiments 1-3, wherein the method does not comprise contacting the T cell with TGF-β.

Embodiment 7 is the method of any one of embodiments 1-3, wherein the method does not comprise contacting the T cell with IL-2 or TGF-β.

Embodiment 8 is the method of any one of embodiments 1-7, wherein the one or more CD3-stimulation agent(s) comprises an effective amount of an anti-CD3 antibody.

Embodiment 9 is the method of any one of embodiments 1-8, wherein the one or more CD3-stimulation agent(s) comprise a methyl transferase inhibitor.

Embodiment 10 is the method of any one of embodiments 3-9, wherein the one or more CD28-stimulation agents comprises an anti-CD28 activating antibody.

Embodiment 11 is the method of any one of embodiments 3-10, wherein the one or more agent(s) that decreases CD28 expression and/or activity comprise an anti-CD28 blocking antibody.

Embodiment 12 is the method of any one of embodiments 2-11, wherein the one or more agent(s) that decreases CD28 expression and/or activity comprise a small interfering RNA (siRNA) or a short hairpin RNA (shRNA).

Embodiment 13 is the method of embodiment 12, wherein the siRNA or the shRNA decreases expression of CD28 in a T cell.

Embodiment 14 is the method of embodiment 13, wherein the siRNA comprises a sequence of one of SEQ ID NOs: 1-6.

Embodiment 15 is the method of embodiment 12, wherein the siRNA or shRNA decreases expression of one or more of p85, p110, PIP3, PKB/Akt, mTOR, IκB, GSK3β, NFκB, NFAT, LCK, FYN, and ITK in a T cell.

Embodiment 16 is the method of any one of embodiments 12-14, wherein the method further comprises introducing into the T cell a nucleic acid construct comprising a sequence encoding the siRNA or the shRNA.

Embodiment 17 is the method of embodiment 16, wherein the nucleic acid construct further comprises a promoter operably linked to the sequence encoding the shRNA.

Embodiment 18 is the method of any one of embodiments 2-17, wherein the one or more agent(s) that decreases CD28 expression and/or activity comprise a small molecule inhibitor of any one of: LCK, FYN, and ITK.

Embodiment 19 is the method of any one of embodiments 2-18, wherein the step of contacting of the T cell with an effective amount of one or more agent(s) that decreases CD28 expression and/or activity further comprises removing the one or more agent(s) that decreases CD28 expression and/or activity after about 1 hour to about 60 hours of the first period of time.

Embodiment 20 is the method of any one of embodiments 3-19, wherein step (a) is performed before step (b) and step (c).

Embodiment 21 is the method of any one of embodiments 3-19, wherein step (c) is performed before step (a) and step (b).

Embodiment 22 is the method of any one of embodiments 3-19, wherein step (b) is performed after step (a) and before step (c).

Embodiment 23 is the method of embodiment 1 or 2, wherein step (a) is performed before step (b).

Embodiment 24 is the method of embodiment 1 or 2, wherein step (b) is performed before step (a).

Embodiment 25 is the method of any one of embodiments 1-24, wherein the method further comprises introducing into the T cell an effective amount of a nucleic acid sequence encoding a truncated nerve growth factor receptor (tNGFR) polypeptide.

Embodiment 26 is the method of any one of embodiment 1-25, wherein the introducing step further comprises introducing into a T cell a nucleic acid construct, wherein the nucleic acid construct comprises the nucleic acid sequence encoding the FOXP3 polypeptide.

Embodiment 27 is the method of embodiment 26, wherein the nucleic acid further comprises a nucleic acid sequence encoding one of the one or more agents that decrease CD28 expression and/or activity.

Embodiment 28 is the method of embodiment 27, wherein the one of the one or more agents that decrease CD28 expression and/or activity is a siRNA or a shRNA.

Embodiment 29 is the method of embodiment 28, wherein the siRNA comprises a sequence of one of SEQ ID NOs: 1-6.

Embodiment 30 is the method of any one of embodiments 26-29, wherein the nucleic acid construct further comprises a promoter operably linked to the nucleic acid sequence encoding the FOXP3 polypeptide.

Embodiment 31 is the method of any one of embodiments 27-30, wherein the nucleic acid construct further comprises a promoter operably linked to the nucleic acid sequence encoding one of the one or more agents that decrease CD28 expression and/or activity.

Embodiment 32 is the method of any one of embodiments 2-31, wherein the introducing step comprises introducing into a T cell a nucleic acid construct, wherein the nucleic acid construct comprises the nucleic acid sequence encoding the FOXP3 polypeptide, a second nucleic acid sequence encoding one of the one or more agents that decrease CD28 expression and/or activity, and a third nucleic acid sequence encoding a tNGFR polypeptide.

Embodiment 33 is the method of any one of embodiments 2-25, wherein the introducing step further comprises introducing into a T cell a nucleic acid construct, wherein the nucleic acid construct comprises the nucleic acid sequence encoding the FOXP3 polypeptide and a second nucleic acid sequence encoding a tNGFR polypeptide.

Embodiment 34 is the method of embodiment 26 or 27, wherein the introducing step further comprises introducing into the T cell a nucleic acid construct comprising a nucleic acid sequence encoding a tNGFR polypeptide.

Embodiment 35 is the method of any one of embodiments 16, 17, and 26-34, wherein the nucleic acid construct comprises a viral vector selected from the group consisting of a lentiviral vector, a retroviral vector, an adenoviral vector, or an adeno-associated viral (AAV) vector.

Embodiment 36 is the method of embodiment 35, wherein the viral vector is a lentiviral vector.

Embodiment 37 is the method of embodiment 35 or 36, wherein the introducing step comprises viral transduction.

Embodiment 38 is the method of any one of embodiments 1-37, wherein the T cell is a CD4+ T cell or a CD4+/CD45RA+ T cell.

Embodiment 39 is the method of any one of embodiments 1-38, wherein the method further comprises, before step (a): obtaining the T cell from a patient or obtaining T cells allogenic to the patient.

Embodiment 40 is the method of embodiment 39, wherein the method further comprises: treating the obtained T cells to isolate a population of cells enriched for CD4+ T cells or CD4+/CD45RA+ T cells.

Embodiment 41 is a T cell produced by the method of any one of embodiments 1-40.

Embodiment 42 is a composition comprising the T cell of embodiment 41.

Embodiment 43 is a T cell comprising: a first nucleic acid sequence encoding a FOXP3 polypeptide; and one or more agents that decreases CD28 expression and/or activity.

Embodiment 44 is the T cell of embodiment 43, wherein the presence of the first nucleic acid sequence and the one or more agents that decreases CD28 expression and/or activity in the T cell induce the T cell to develop or further develop one or more characteristics of a T regulatory phenotype.

Embodiment 45 is the T cell of embodiment 43 or 44, wherein the T cell further comprises a third nucleic acid sequence encoding a tNGFR polypeptide.

Embodiment 46 is a T cell comprising a first nucleic acid sequence encoding a FOXP3 polypeptide; and a second nucleic acid sequence encoding a tNGFR polypeptide.

Embodiment 47 is the T cell of embodiment 46, wherein the presence of the first nucleic acid sequence and the second nucleic acid sequence in the T cell induce the T cell to develop or further develop one or more characteristics of a T regulatory phenotype.

Embodiment 48 is the T cell of any one of embodiments 43-45, wherein the one or more agents that decreases CD28 expression and/or activity comprises a small interfering RNA (siRNA) or a short hairpin RNA (shRNA).

Embodiment 49 is the T cell of embodiment 48, wherein the siRNA or the shRNA decreases expression of CD28 in a mammalian cell.

Embodiment 50 is the T cell of embodiment 48, wherein the siRNA or shRNA decreases expression of one or more of p85, p110, PIP3, PKB/Akt, mTOR, IκB, GSK3β, NFκB, NFAT, LCK, FYN, and ITK in a T cell.

Embodiment 51 is the T cell of embodiment 50, wherein the siRNA comprises a sequence of one of SEQ ID NOs: 1-6.

Embodiment 52 is a composition comprising a T cell of any one of embodiments 43-51.

Embodiment 53 is a method of producing a T cell population expressing an exogenous FOXP3 polypeptide and a siRNA, the method comprising culturing a T cell of any one of embodiments 43-51 in growth media under conditions sufficient to expand the population of T cells.

Embodiment 54 is a population of T cells prepared by the method of embodiment 53.

Embodiment 55 is a composition comprising the population of T cells of embodiment 54.

Embodiment 56 is a vector comprising a first nucleic acid sequence encoding a FOXP3 polypeptide and a second nucleic acid sequence encoding a siRNA or a shRNA that decreases CD28 expression and/or activity.

Embodiment 57 is the vector of embodiment 56, wherein the first nucleic acid sequence is operably linked to a promoter, the second nucleic acid sequence is operably linked to a promoter, or the first nucleic acid sequence and the second nucleic acid sequence are both operably linked to a promoter.

Embodiment 58 is the vector of embodiment 57, wherein the presence of the first nucleic acid sequence and the second nucleic acid sequence in the T cell induce the T cell to develop or further develop one or more characteristics of a T regulatory phenotype.

Embodiment 59 is a vector comprising a first nucleic acid sequence encoding a FOXP3 polypeptide and a second nucleic acid sequence encoding a tNGFR polypeptide.

Embodiment 60 is the vector of embodiment 59, wherein the first nucleic acid sequence is operably linked to a promoter, the second nucleic acid sequence is operably linked to a promoter, or the first nucleic acid sequence and the second nucleic acid sequence are both operably linked to a promoter.

Embodiment 61 is the vector of embodiment 60, wherein the presence of the first nucleic acid sequence and the second nucleic acid sequence in the T cell induce the T cell to develop or further develop one or more characteristics of a T regulatory phenotype.

Embodiment 62 is a vector comprising a first nucleic acid sequence encoding a FOXP3 polypeptide, a second nucleic acid sequence encoding a siRNA or a shRNA that decreases CD28 expression and/or activity, and a third nucleic acid sequence encoding a tNGFR polypeptide.

Embodiment 63 is the vector of embodiment 62, wherein the first nucleic acid sequence is operably linked to a promoter, the second nucleic acid sequence is operably linked to a promoter, the third nucleic acid sequence is operably linked to a promoter, or the first nucleic acid sequence, the second nucleic acid sequence and the third nucleic acid sequence are all operably linked to a promoter.

Embodiment 64 is the vector of embodiment 62, wherein the presence of the first nucleic acid sequence, the second nucleic acid sequence, and the third nucleic acid sequence in the T cell induce the T cell to develop or further develop one or more characteristics of a T regulatory phenotype.

Embodiment 65 is the vector of any one of embodiments 56-58 and 62-64, wherein the siRNA or the shRNA decreases expression of CD28 in a T cell.

Embodiment 66 is the vector of embodiment 65, wherein the siRNA comprises a sequence of one of SEQ ID NOs: 1-6.

Embodiment 67 is the vector of any one of embodiments 56-58 and 62-64, wherein the siRNA or shRNA decreases expression of one or more of p85, p110, PIP3, PKB/Akt, mTOR, IκB, GSK3β, NFκB, NFAT, LCK, FYN, and ITK in a T cell.

Embodiment 68 is the vector of any one of embodiments 56-67, wherein the vector comprises a viral vector selected from the group consisting of a lentiviral vector, a retroviral vector, an adenoviral vector, or an adeno-associated viral (AAV) vector.

Embodiment 69 is the vector of embodiment 68, wherein the viral vector is a lentiviral vector.

Embodiment 70 is a composition comprising the vector of any one of embodiments 56-69.

Embodiment 71 is a kit comprising the composition of any one of embodiments 42, 52, 55, and 70.

Embodiment 72 is a method of treating an autoimmune disease or disorder in a patient comprising administering a T cell of any one of embodiments 41 and 43-51, or a composition of any one of embodiments 42, 52, 55, and 70.

Embodiment 73 is the method of embodiment 72, wherein the subject is previously diagnosed or identified as having an autoimmune disease or disorder.

Embodiment 74 is the method of embodiment 73, wherein the autoimmune disease or disorder is Lupus, Rheumatoid Arthritis, Multiple Sclerosis, Insulin Dependent Diabetes Mellitis, Myasthenia Gravis, Graves disease, Autoimmune Hemolytic Anemia, Autoimmune Thrombocytopenia Purpura, Goodpasture's Syndrome, Pemphigus Vulgaris, acute Rheumatic Fever, Post-Streptococcal Glomerulonephritis, Crohn's Disease, Celiac Disease, or Polyarteritis Nodosa.

Embodiment 75 is the method of embodiment 72, wherein administering the autologous or allogenic T cell population comprises intravenous injection or intravenous infusion.

Embodiment 76 is the method of embodiment 72, wherein the administering results in amelioration of one or more symptoms of the autoimmune disease or disorder.

Embodiment 77 is a method of transducing a T cell, comprising: (a) contacting a T cell with an effective amount of (i) one or more CD3-stimulation agent(s) in the absence of a CD28 stimulating agent for a first period of time under conditions that allow for stimulation and activation of the T cell, and (b) introducing into the T cell an effective amount of a nucleic acid sequence encoding one or more polypeptides operatively linked to a promoter active in T cells, thereby transducing the T cell.

Embodiment 78 is the method of embodiment 77, further comprising contacting the T cell with an effective amount of one or more agent(s) that decreases CD28 expression and/or activity.

Embodiment 79 is a method of transducing a T cell, comprising: (a) contacting a T cell with an effective amount of (i) one or more CD3-stimulation agent(s), and (ii) one or more CD28-stimulation agent(s) for a first period of time under conditions that allow for stimulation and activation of the T cell; (b) contacting the T cell with an effective amount of one or more agent(s) that decreases CD28 expression and/or activity; and (c) introducing into the T cell an effective amount of a nucleic acid sequence encoding one or more polypeptides operatively linked to a promoter active in T cells, thereby transducing the T cell.

Embodiment 80 is the method of any one of embodiments 77-79, wherein the one or more CD3-stimulation agent(s) comprises an effective amount of an anti-CD3 antibody.

Embodiment 81 is the method of any one of embodiments 77-80, wherein the one or more CD3-stimulation agent(s) comprise a methyl transferase inhibitor.

Embodiment 82 is the method of any one of embodiments 79-81, wherein the one or more CD28-stimulation agents comprises an anti-CD28 activating antibody.

Embodiment 83 is the method of any one of embodiments 79-82, wherein the one or more agent(s) that decreases CD28 expression and/or activity comprise an anti-CD28 blocking antibody.

Embodiment 84 is the method of any one of embodiments 78-83, wherein the one or more agent(s) that decreases CD28 expression and/or activity comprise a small interfering RNA (siRNA) or a short hairpin RNA (shRNA).

Embodiment 85 is the method of embodiment 84, wherein the siRNA or the shRNA decreases expression of CD28 in a T cell.

Embodiment 86 is the method of embodiment 85, wherein the siRNA comprises a sequence of one of SEQ ID NOs: 1-6.

Embodiment 87 is the method of embodiment 84, wherein the siRNA or shRNA decreases expression of one or more of p85, p110, PIP3, PKB/Akt, mTOR, IκB, GSK3β, NFκB, NFAT, LCK, FYN, and ITK in a T cell.

Embodiment 88 is the method of any one of embodiments 84-86, wherein the method further comprises introducing into the T cell a nucleic acid construct comprising a sequence encoding the siRNA or the shRNA.

Embodiment 89 is the method of embodiment 88, wherein the nucleic acid construct further comprises a promoter operably linked to the sequence encoding the shRNA.

Embodiment 90 is the method of any one of embodiments 78-89, wherein the one or more agent(s) that decreases CD28 expression and/or activity comprise a small molecule inhibitor of any one of: LCK, FYN, and ITK.

Embodiment 91 is the method of any one of embodiments 78-90, wherein the step of contacting of the T cell with an effective amount of one or more agent(s) that decreases CD28 expression and/or activity further comprises removing the one or more agent(s) that decreases CD28 expression and/or activity after about 1 hour to about 60 hours of the first period of time.

Embodiment 92 is the method of any one of embodiments 79-91, wherein step (a) is performed before step (b) and step (c).

Embodiment 93 is the method of any one of embodiments 79-91, wherein step (c) is performed before step (a) and step (b).

Embodiment 94 is the method of any one of embodiments 79-91, wherein step (b) is performed after step (a) and before step (c).

Embodiment 95 is the method of embodiment 77 or 78, wherein step (a) is performed before step (b).

Embodiment 96 is the method of embodiment 77 or 78, wherein step (b) is performed before step (a).

Embodiment 97 is the method of any one of embodiments 77-96, wherein the one or more polypeptides is one more exogenous polypeptides.

Embodiment 98 is the method of any one of embodiments 77-97, wherein one of the one or more polypeptides is a chimeric antigen receptor.

Embodiment 99 is the method of embodiment 98, wherein the chimeric antigen receptor comprises an antigen-binding domain capable of binding to CD19.

Embodiment 100 is the method of embodiment 99, wherein the nucleic acid further comprises a nucleic acid sequence encoding one of the one or more agents that decrease CD28 expression and/or activity.

Embodiment 101 is the method of embodiment 100, wherein the one of the one or more agents that decreases CD28 expression and/or activity is a siRNA or a shRNA.

Embodiment 102 is the method of embodiment 101, wherein the siRNA comprises a sequence of one of SEQ ID NOs: 1-6.

Embodiment 103 is the method of any one of embodiments 88-96, wherein the nucleic acid construct further comprises a promoter that is operably linked to the sequence encoding the siRNA or the shRNA.

Embodiment 104 is the method of any one of embodiments 88 or 89, wherein the nucleic acid construct comprises a viral vector selected from the group consisting of a lentiviral vector, a retroviral vector, an adenoviral vector, or an adeno-associated viral (AAV) vector.

Embodiment 105 is the method of any one of embodiments 77-104, wherein the nucleic acid sequence encoding one or more polypeptides operatively linked to the promoter active in T cells is present in a viral vector selected from the group consisting of a lentiviral vector, a retroviral vector, an adenoviral vector, or an adeno-associated viral (AAV) vector.

Embodiment 106 is the method of embodiment 104 or 105, wherein the viral vector is a lentiviral vector.

Embodiment 107 is the method of any one of embodiments 104-106, wherein the introducing step comprises viral transduction.

Embodiment 108 is the method of any one of embodiments 77-107, wherein the T cell is a CD4⁺ T cell or a CD4⁺/CD45RA⁺ T cell.

Embodiment 109 is the method of any one of embodiments 77-108, wherein the method further comprises, before step (a): obtaining the T cell from a patient or obtaining T cells allogenic to the patient.

Embodiment 110 is the method of embodiment 109, wherein the method further comprises: treating the obtained T cells to isolate a population of cells enriched for CD4+ T cells or CD4+/CD45RA+ T cells.

Embodiment 111 is a T cell produced by the method of any one of embodiments 77-110.

Embodiment 112 is a composition comprising the T cell of embodiment 111 and a pharmaceutically acceptable carrier.

Embodiment 113 is a method of treating a subject in need thereof with the T-cell of embodiment 111 or the composition of embodiment 112.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of producing a T regulatory cell, comprising:
(a) contacting a T cell with an effective amount of one or more CD3-stimulation agent(s) in the absence of (i) a CD28 stimulating agent and (ii) IL-2, for a first period of time under conditions that allow for stimulation and activation of the T cell, and
(b) after (a), transducing into the T cell a viral vector comprising a nucleic acid sequence encoding a forkhead box protein 3 (FOXP3) polypeptide,
wherein the presence of the nucleic acid sequence in the T cell induces the T cell to develop or further develop one or more characteristics of a T regulatory cell phenotype compared to when the nucleic acid sequence is not present in the T cell.

2. The method of claim 1, wherein the method further comprises contacting the T cell with an effective amount of one or more agent(s) that decreases CD28 expression and/or activity.

3. The method of claim 1, wherein the method does not comprise contacting the T cell with TGF-β.

4. The method of claim 1, wherein the one or more CD3-stimulation agent(s) comprises an effective amount of an anti-CD3 antibody.

5. The method of claim 1, wherein the one or more CD3-stimulation agent(s) comprise a methyl transferase inhibitor.

6. The method of claim 2, wherein the one or more agent(s) that decreases CD28 expression and/or activity comprise an anti-CD28 blocking antibody.

7. The method of claim 2, wherein the one or more agent(s) that decreases CD28 expression and/or activity comprise a small interfering RNA (siRNA) or a short hairpin RNA (shRNA).

8. The method of claim 7, wherein the siRNA or the shRNA decreases expression of CD28 in a T cell.

9. The method of claim 8, wherein the siRNA comprises a sequence of one of SEQ ID NOs: 1-6.

10. The method of claim 7, wherein the siRNA or shRNA decreases expression of one or more of p85, p110, PIP3, PKB/Akt, mTOR, IκB, GSK3β, NFκB, NFAT, LCK, FYN, and ITK in a T cell.

11. The method of claim 7, wherein the method further comprises introducing into the T cell a nucleic acid construct comprising a sequence encoding the siRNA or the shRNA.

12. The method of claim 11, wherein the nucleic acid construct further comprises a promoter operably linked to the sequence encoding the shRNA.

13. The method of claim 2, wherein the one or more agent(s) that decreases CD28 expression and/or activity comprise a small molecule inhibitor of any one of: LCK, FYN, and ITK.

14. The method of claim 1, wherein the viral vector further comprises a nucleic acid sequence encoding one of the one or more agents that decrease CD28 expression and/or activity.

15. The method of claim 14, wherein the one of the one or more agents that decrease CD28 expression and/or activity is a siRNA or a shRNA.

16. The method of claim 15, wherein the siRNA comprises a sequence of one of SEQ ID NOs: 1-6.

17. The method of claim 1, wherein the viral vector further comprises a promoter operably linked to the nucleic acid sequence encoding the FOXP3 polypeptide.

18. The method of claim 14, wherein the viral vector further comprises a promoter operably linked to the nucleic acid sequence encoding one of the one or more agents that decrease CD28 expression and/or activity.

19. The method of claim 1, wherein the viral vector is selected from the group consisting of: a lentiviral vector, a retroviral vector, an adenoviral vector, and an adeno-associated viral (AAV) vector.

20. The method of claim 19, wherein the viral vector is a lentiviral vector.

21. The method of claim 1, wherein the T cell is a CD4+ T cell or a CD4+/CD45RA+ T cell.

22. The method of claim 1, wherein the method further comprises, before step (a):
obtaining the T cell from a patient.

23. The method of claim 22, wherein the method further comprises, after step (b), administering the T regulatory cell to the subject.
